(19) United States
(12) Patent Application Publication
(10) Pub. No.: US 2022/0151921 A1
(43) Pub. Date: May 19, 2022

(54) MONOCYTE-SELECTIVE DRUG DELIVERY SYSTEM USING SINGLE-WALLED CARBON NANOTUBES TO INDUCE EFFEROCYTOSIS

(71) Applicant: The Board of Trustees of the Leland Stanford Junior University, Stanford, CA (US)

(72) Inventors: Niloufar Hoesseini Nassab, Stanford, CA (US); Bryan Ronain Smith, East Lansing, MI (US); Nicholas James Leeper, Stanford, CA (US); Alyssa Monica Flores, Martinez, CA (US); Jianqin Ye, San Francisco, CA (US)

(21) Appl. No.: 17/437,370

(22) PCT Filed: Mar. 11, 2020
(86) PCT No.: PCT/US2020/022092

§ 371 Date: Sep. 8, 2021

Related U.S. Application Data
(60) Provisional application No. 62/819,443, filed on Mar. 15, 2019.

(51) Int. Cl. A61K 9/00; A61K 31/47

(52) U.S. Cl. CPC A61K 9/0092; B82Y 5/00; A61K 31/47

(57) ABSTRACT

Abstract: Provided herein are compositions and methods of using single-walled carbon nanotubes (SWNTs) to target a specific subset of immune calls bearing an anti-phagocytic signal for selective delivery of a therapeutic agent (e.g a small molecule inhibitor of an anti-phagocytic signal) to inhibit the “don't-eat-me” signaling pathway and restore efferocytosis. For example, using SWNTs, an inhibitor of SHP1 phosphatase (“SHP1i”) acting in the CD47/SIRPa signaling pathway was specifically delivered to Ly-6Chi monocytes that avoid efferocytosis and home to atherosclerotic plaques allowing apoptotic cells to accumulate. The targeted delivery of SHP1i to these monocytes resulted in an increase in efferocytosis and the stabilization and reduction in atherosclerotic plaques. These compositions and methods can be used in the treatment of inflammatory diseases or disorders, such as, for example, atherosclerosis.

### CROSS REFERENCE

This application claims benefit of U.S. Provisional Patent Application No. 62/819,443, filed Mar. 15, 2019, which applications are incorporated herein by reference in their entirety.

### TECHNICAL FIELD

The present disclosure is directed to the field of targeted drug delivery using single-walled carbon nanotubes (SWNTs). Provided herein are compositions and methods for treating inflammatory diseases or disorders, such as, for example, atherosclerosis, using SWNTs to target a specific subset of immune cells for selective delivery of a therapeutic agent (e.g an inhibitor of an anti-phagocytic signal), thereby stimulating efferocytosis.

### BACKGROUND

Each day, more than 100 billion cells die in the human body. Dead or dying cells can be swiftly and efficiently cleared from the body via a phagocytic process known as efferocytosis to prevent the inflammatory consequences associated with the accumulation of apoptotic debris. Efferocytosis can be suppressed by cell-surface expression of anti-phagocytic “dont eat me” signals such as CD47.

Interestingly, CD47 is upregulated significantly in atherosclerotic cardiovascular disease (ASCVD), as well as in many cancers, and studies have shown that defective efferocytosis plays a role in atherosclerosis. At least because overexpression of the CD47 anti-phagocytic signal was found in subjects with ASCVD, CD47 is an attractive target for pro-efferocytic therapies that block or inhibit the anti-phagocytic signal.

ASCVD is a complex disease involving multiple biological pathways, including the immune system and inflammatory response, and remains the primary cause of morbidity and mortality worldwide. Despite appropriate evidence-based treatments for patients with ASCVD, recurrence and mortality rates remain at approximately 2-4% per year.

Risk factors for ASCVD can sometimes be attributed to genetic background and environmental factors, which together can lead to individual variations in response to therapy. Atherosclerotic disease is also influenced by the complex nature of the cardiovascular system itself where anatomy, function and biology all play important roles in health as well as disease.

Atherosclerotic plaque consists of accumulated intracellular and extracellular lipids, smooth muscle cells, connective tissue, and glycosaminoglycans. The earliest detectable lesion of atherosclerosis is the fatty streak, consisting of lipid-laden foam cells, which are macrophages that have migrated as monocytes from the circulation into the subendothelial layer of the intima, which later evolves into the fibrous plaque, consisting of intimal smooth muscle cells surrounded by connective tissue and intracellular and extracellular lipids.

Therapies which re-establish normal efferocytosis and result in the clearance of apoptotic debris have great potential for treating immune and/or inflammatory diseases including ASCVD, coronary artery disease (CAD), atherosclerosis and the like. For example, anti-CD47 therapies such as the use of antibodies were found to restore efferocytosis, but also resulted in many side effects, such as anemia and hyperbilirubinemia. Thus, a need remains for strategies for selective delivery to the target pariculary subsets of monocytes and macrophages to achieve the same therapeutic efficacy without the side effects.

Carbon nanotubes have a wide range of commercial uses, such as, for example, in electronic devices, as electromechanical actuators, electrochemical sensors, cancer imaging and drug delivery systems, to name but a few applications. In animals, single-walled carbon nanotubes (SWNTs) functionalized by PEGylated phospholipids have been shown to be non-toxic over a period of at least 144 days, and have been developed for possible use in high performance in vivo near-IR (>1 μm) imaging and photothermal cancer therapy (Robinson et al., 2010, Nano Res. 3(11):779-793).

A need remains for compositions and methods for specific delivery of therapeutic agents targeted to particular tissues and/or cells for treatment of diseases in mammalian subjects, including humans. Methods employing minimally-invasive monocyte-selective targeted therapies for treatment or prevention of immune or inflammatory diseases or disorders such as atherosclerosis have distinct advantages over more costly and more invasive treatments such as the use of stents and surgery. Furthermore, selective delivery of therapeutic agents can reduce the effective dosage of the agent, and reduce potential side effects and/or toxicity that may be observed when the drug is introduced or administered systemically and/or at higher dosage.

### SUMMARY OF THE INVENTION

Herein disclosed are compositions and methods for use as targeted delivery system employing single-walled carbon nanotubes (SWNTs) to direct a therapeutic agent in a highly selective manner to monocytes/macrophages. SWNTs are almost exclusively taken up by a single immune cell subset, Ly-6C^{hi} monocytes. These monocytes naturally home to atherosclerotic plaques in mice. With the present disclosure, the CD47-SIRPα pathway is inhibited by employing SWNTs to deliver a small molecule inhibitor of a downstream tyrosine phosphatase enzyme, SHP1, to tissues marked by the presence of Ly-6C^{hi} monocytes. Thus, SWNTs selectively deliver the small molecule inhibitor to a subset of immune cells in an advanced immune-based delivery strategy. This targeted drug delivery system can remarkably reduce the required dosage of the drug, selectively deliver the drug to the target area, and minimize drug-associated side effects. Moreover, because a lower dosage of drug is needed and no antibody is required, it is likely to be more cost effective.

In some embodiments compositions are provided that comprise single-walled carbon nanotubes (SWNTs) and a therapeutic agent. In some embodiments of the composition, the therapeutic agent is a small molecule. In some embodiments of the composition, the small molecule is an inhibitor of a molecule in a CD47/SIRPα signaling pathway. In some embodiments of the composition, the small molecule inhibits SHP1 phosphatase. In some embodiments of the composition, the small molecule is the SHP1 inhibitor TPI-1. In some embodiments of the composition, the small molecule that inhibits SHP1 is NSC-87877. In some embodiments, the therapeutic agent inhibits SHP1 and increases efferocytosis in cardiovascular cells, including, for example, smooth muscle cells. The small molecule may be joined to the SWNTS in a covalent or non-covalent manner. In some embodiments a non-covalent binding, e.g. π-π stacking, is employed. An advantage of π-π stacking can be a pH dependent release of the small molecule from the SWNTS.

In some embodiments, the compositions provide for targeted delivery of a therapeutic agent that increases efferocytosis in a diseased tissue marked by the presence of Ly-6C^{hi} monocytes. The SWNTs deliver the therapeutic agent for delivery to and treatment of an immune or inflammatory disorder. In some embodiments, the immune or inflammatory disorder is an atherosclerotic cardiovascular disease (ASCVD). In some embodiments of the composition, the therapeutic agent increases efferocytosis of apoptotic cells or cellular degradation products by reversing an anti-phagocytic signal. In some embodiments of the composition, the therapeutic agent increases efferocytosis of cellular degradation products in atherosclerotic plaques.

In other embodiments, methods are provided for the prevention and treatment of immune and/or inflammatory disorders including without limitation atherosclerotic cardiovascular disease (ASCVD) or coronary artery disease (CAD) in a subject. The methods of the invention are based in part on the finding that apoptotic smooth muscle cells (SMC) in carriers of the risk allele can be resistant to efferocytosis, leading to the retention of such cells in the necrotic core of atherosclerotic plaque. In the methods of the invention, a SWNTS composition comprising a therapeutic agent that increases efferocytosis of cellular components of coronary plaque, including efferocytosis of apoptotic smooth muscle cells, is administered to the subject in a dose and for a period of time effective to stabilize, prevent or reduce the immune and/or inflammatory condition in the individual. Methods include without limitation methods of preventing or treating atherosclerosis, stabilizing plaques, and reducing plaque formation.

In some embodiments, the subject is homozygous or heterozygous for a 9p21 risk allele. In some such embodiments the methods include genetic testing of the subject for the presence of a 9p21 risk allele. In other such embodiments the subject has been previously diagnosed for the presence of a 9p21 risk allele, where such methods may include, without limitation, analyzing a sample of genomic DNA from the individual for the presence of sequences of human chromosome 9p21 associated risk of CAD, including SNPs associated with the risk locus. In some embodiments of the method, the 9p21 risk allele is genotyped by determination of the presence of an SNP variant at 9p21 associated with risk. In some embodiments of the method, the subject is a mammal. In some embodiments of the method, the mammal is a human.

Molecular targets for increasing efferocytosis include, without limitation, agents that inhibit signaling in the the anti-phagocytic CD47-SIRPα signaling axis; agents that inhibit the activity of the SHP1 phosphatase; agents that block the interaction of CD47 and SIRPαand

In some embodiments of the method, the immune and/or inflammatory disorder is ASCVD. In some embodiments of the method, the therapeutic agent increases efferocytosis of apoptotic cells or cellular degradation products by reversing an anti-phagocytic signal. In some embodiments of the method, the therapeutic agent increases efferocytosis of cellular degradation products in atherosclerotic plaques. In some embodiments of the method, the therapeutic agent is a small molecule. In some embodiments of the method, the small molecule is an inhibitor of a molecule in a CD47/SIRPa signaling pathway.

In some aspects, provided herein is a kit for delivery of a therapeutic agent that increases efferocytosis to treat an immune and/or inflammatory disorder in a mammalian subject, comprising single-walled carbon nanotubes (SWNTs) comprising a therapeutic agent that increases efferocytosis for delivery to and treatment of an individual having an immune or inflammatory disorder; and instructions for use.

In some aspects, disclosed herein is a use of the compositions described herein in the manufacture of a medicament to stabilize, prevent or reduce atherosclerotic plaques, wherein the medicament is used to treat an immune and/or inflammatory disorder in a mammalian subject.

In some embodiments, the therapeutic agent is an efferocytosis stimulating agent, and the agent is used in the manufacture of a medicament to stabilize, prevent or reduce atherosclerotic plaque, wherein the medicament is administered to an individual having or at risk of having atherosclerosis.

Still another aspect of the present invention provides a kit to stabilize, prevent or reduce atherosclerotic plaque. The kit includes an efferocytosis stimulating agent, in an amount sufficient to stabilize, prevent or reduce atherosclerotic plaque. The kit may also include reagents for genotyping at human chromosome 9p21, including alleles of rs10757278 and rs1333049. The kit may also instructions for use, reagents for monitoring atherosclerotic disease, and the like.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1F: SWNT-SHP1i promotes the phagocytosis of apoptotic cells by macrophages. FIG. 1A Schematic of SWNT-SHP1i, comprised of a backbone of single-walled carbon nanotubes (SWNTs) which are functionalized with phospholipid-polyethene glycol (PEG) to form biocompatible nanotubes, Cy5.5 fluorophore for tracking in vivo delivery, and small-molecule inhibitors of SHP-1 (SHP1i) via π-π stacking on the nanotube surface. FIG. 1B UV-vis spectrum of SWNTs (black), SWNT-Cy5.5 (blue), and SWNT-SHP1i (red). FIG. 1C Release curve of SHP1i from SWNT-Cy5.5 in serum, demonstrating controlled release over 7 days. FIG. 1D, 1E Cellular uptake assays demonstrate the targeting ability of SWNTs for murine macrophages compared to endothelial cells and VSMCs. FIG. 1A DFlow cytometry histograms of cells from uptake studies with SWNT-Cy5.5, plain SWNTs (not adorned with Cy5.5), and PBS controls. M_{T}, macrophages. FIG. 1E. FIG. 1F In vitro phagocytosis assays confirm that SWNT-SHP1i augments the clearance of apoptotic vascular cells by macrophages at least as potently as gold standard anti-CD47 antibodies, compared with SHP1i and SWNT controls. Error bars represent s.e.m. *p<0.05. ***p<0.001. ****p<0.0001 by one-way ANOVA with a Tukey post hoc test or by an unpaired two-tailed t-test.

FIG. 2A-2F: Vascular-tropic SWNTs home to phagocytes in the atherosclerotic plaque. FIG. 2A Time-interval biodistribution studies demonstrate that SWNTs primarily accumulate in the atherosclerotic aortae after intravenous injection. FIG. 2B Biodistribution profile at 7 days after administration of SWNTs reveals that SWNTs are cleared from the blood pool (as indicated by low Cy5.5 fluorescence in circulating white blood cells) and do not chronically accumulate in the spleen or other visceral organs. FIG. 2C, 2D SWNTs home to Ly-6Chi monocytes and macrophages in the aorta, while SWNT detection is low in other vascular cells following four weeks of weekly SWNT administration. FIG. 2E Confocal imaging confirms that SWNTs co-localize with lesional macrophages in the aortic sinus (red), as indicated by co-staining for PEG on PEG-functionalized SWNTs (green). FIG. 2F At 48 hr post-injection, SWNTs home to macrophages in the aorta and are not found at high levels in macrophages of the spleen. Error bars represent s.e.m. *p<0.05, **p<0.01, ****p<0.0001 by one-way ANOVA with a Tukey post hoc test.

FIG. 3A-3F: Pro-efferocytic SWNTs prevent atherosclerosis. FIG. 3A Mice treated with SWNT-SHP1i (n=19) develop significantly reduced plaque content in the aortic sinus, relative to SWNT-Cy5.5 controls (n=17). FIG. 3B SWNT-SHP1i decreases phosphorylation of SHP-1, indicating silencing of the anti-phagocytic CD47-SIRPα signal. FIG. 30-3E Lesions from mice treated with pro-efferocytic SWNTs are more likely to have apoptotic cells (indicated by arrows) that have been ingested by lesional macrophages FIG. 3C develop smaller necrotic cores (indicated by dotted lines) (d), and accumulate less apoptotic debris (as assessed by percentage of cleaved caspase-3+area in the plaque). FIG. 3E. FIG. 3F ¹⁸F-FDG PET/CT imaging demonstrates that SWNT-SHP1i significantly reduces vascular inflammation. SUVmean, mean standardized uptake value. Error bars represent s.e.m. *p<0.05, **p<0.01 by the Mann-Whitney U test or unpaired two-tailed t-test.

FIG. 4A-4D: Single-cell transcriptomics reveal genes and key molecular pathways modulated by chronic CD47-SIRPα blockade in lesional macrophages. FIG. 4A Workflow for scRNA-seq including aortic cell isolation, drop-sequencing, and downstream analyses. FIG. 4B Unsupervised dimensionality reduction identifies 7 major cell types with similar gene expression from the combined SWNT-Cy5.5 control and SWNT-SHP1i datasets. Data is visualized using t-distributed stochastic neighbor embedding (t-SNE) plots, showing the 7 distinct cell clusters (left), and SWNT detection in each cell (right). SWNT-positive cells are the most prevalent in lesional macrophages (Cluster 1) and macrophage-like cells (Cluster 5, FIG. 9). Memory Tc, memory T cells; DCs, dendritic cells; M_{T}-like, macrophage-like; CD4+/CD8+Tc, CD4+/CD8+T cells. FIG. 4C Heatmap showing gene expression of 10 cluster-defining genes and leukocyte markers. FIG. 4D Single-cell differential gene expression analysis identifies the genes regulated by SWNT-SHP1i specifically in lesional macrophages. Gene Ontology (GO) enrichment and pathway analyses reveal that CD47-SIRPα blockade results in an increase in expression of genes related to antigen processing and presentation, and the downregulation of genes associated with monocyte chemotaxis, chemokine signaling, and the cellular response to the pro-inflammatory cytokines, interleukin-1 (IL-1) and interferon-γ (IFN-γ). The subclasses of the top GO biological processes (fold enrichment>10, adjusted p-value<10-2) are shown. Size of circles are proportional to the enrichment of each biological process.

FIG. 5A-5C. Pro-efferocytic SWNTs do not induce clearance of healthy tissue. a,b, Mice treated with SWNT-SHP1i do not develop anemia FIG. 5A or a compensatory reticulocytosis FIG. 5B, which occurs in response to anti-CD47-antibody treatment due to the off-target elimination of red blood cells. FIG. 5C No significant difference is observed for the weight of the spleen between groups, suggestive of the lack of red blood clearance due to erythrophagocytosis. IgG and anti-CD47 antibody data in FIG. 5A and 5B have been reported (Kojima, et al., 2016) **p<0.01, ****p<0.0001 by unpaired two-tailed t-test.

FIG. 6A-6D: FIG. 6A Schematic illustrating steps of SWNT-SHP1i preparation. Following SWNT-PEG-Cy5.5 (SWNT-Cy5.5) fabrication, SHP1i is loaded onto SWNT-Cy5.5 by adding SHP1i to a stirred solution of SWNT-Cy5.5 overnight at 4° C. and removing free SHP1i molecules by dialyzing with PBS for 24 h at 4° C. FIG. 6B Transmission electron microscope image of SWNTs (yellow arrow) and nanoparticle catalyst (white arrow). FIG. 6C Photo depicting color change in solution of SWNTs loaded with SHP1i (left) or without (right). FIG. 6D Release rates of SHP1i from SWNT-Cy5.5 in PBS (pH=7.4).

FIG. 7A-71: FIG. 7A-7C In vitro uptake studies show that SWNTs are preferentially taken up by human and mouse macrophages after 3 hr incubation. Uptake studies are shown in human macrophages (PMA-differentiated THP-1 cells), human aortic endothelial cells (HAECs), and human coronary artery smooth muscle cells (HCASMCs) FIG. 7A-7B, as well as murine macrophages (RAW264.7), endothelial cells (C166), and primary aortic vascular smooth muscle cells (VSMCs) FIG. 7C. FIG. 7D SWNTs show a similarly high targeting efficiency for LPS-stimulated RAW264.7 cells, indicating robust uptake in the activated M1-polarized macrophage phenotype. FIG. 7E Representative flow cytometry plots and staining controls for the conditions of the in vitro phagocytosis assays. Double-positive cells in the right upper quadrant represent macrophages that have ingested a target apoptotic cell. FIG. 7F The phagocytosis efficiency of macrophages (CellTracker Red⁺) against apoptotic vascular cells (CellTracker Orange⁺) is enhanced by SWNT-SHP1i nanoparticles, relative to SWNTs, SWNT-Cy5.5 and SHP1i controls. FIG. 7G Cell viability assays indicate that SWNTs do not affect the viability of RAW264.7 cells, suggesting the absence of a toxic effect on macrophages. PBS served as control. FIG. 7H MTT assays show that SWNT-SHP1i has no effect on the proliferation rates of RAW264.7 macrophages in the presence of 10% serum. FIG. 7I SWNT-SHP1i treatment does not alter the rates of programmed cell death of RAW264.7 macrophages in vitro, as shown by the lack of a difference in TUNEL (terminal deoxynucleotidyl transferase [TdT] dUTP nick-end labeling) staining. Error bars represent s.e.m. *p<0.05, **p<0.01, ***p<0.001, ****p<0.0001 by unpaired two-tailed t-test or one-way ANOVA with a Tukey post hoc test.

FIG. 8A-8H: FIG. 8A Biodistribution studies by flow cytometry reveal greater uptake of SWNT-Cy5.5 in the atherosclerotic aorta at 48 hr post-injection (indicated by greater Cy5.5 fluorescence), as compared to the spleen and liver. FIG. 8B-8C Immunofluorescence confocal imaging show SWNT (immunostained for PEG) accumulation in the aortic sinus FIG. 8B and no signal in the heart FIG. 8C of apoE^{-/-} mice. FIG. 8D There is significantly greater SWNT accumulation in Ly-6C^{hi} monocytes of the aorta than the spleen in the four-week atherosclerosis intervention model. FIG. 8E Similar to results of SWNT-Cy5.5 cell-selectivity (FIG. 2c-d), SHP1i-loaded SWNTs show high targeting efficiency 1 of Ly-6C^{hi} monocytes and macrophages in the aorta, while <10% of other vascular cells contain SWNTs. FIG. 8F Representative flow cytometry plots from in vivo cellular specificity studies. FIG. 8G-8H Histological analysis of lesions in the aortic sinus area show that SWNT-SHP1i results in a significant reduction in plaque area in both male FIG. 8G and female FIG. 8H mice, as measured by Oil Red O (ORO) staining. This finding is particularly important given the widely reported sex-dependent effects on atherosclerosis mouse models that is also relevant to human disease. 1*p<0.05, **p<0.01 by unpaired two-tailed t-test or one-way ANOVA with a Tukey post hoc test.

FIG. 9A-9F: FIG. 9A Flow cytometry gating strategy for selection of viable (SYTOXBlue-) cells that had taken up SWNTs (Cy5.5+). FIG. 9B Sequencing data quality metrics for cells isolated from aortae of mice following treatment with SWNT-Cy5.5 or SWNT-SHP1i. FIG. 9C Violin plots showing number of genes (nGene), unique molecular identifier (nUMI), and percentage of mitochondrial gene reads (percent.mito) for cells in the full dataset. FIG. 9D Scatterplot of nGene and nUMI across the combined dataset used to identify and exclude outliers (e.g. cell doublets). FIG. 9E Representative violin plots showing the distribution of gene expression of immune cell markers in the 7 identified leukocyte clusters. The identity of clusters was defined according to canonical hematopoietic-lineage and immune cell markers: macrophages (Adgre1 encoding F4/80, Cd68, Csf1r), memory T cells (Cd3g, II2r, Ptprc and II7r encoding memory markers CD45RO and CD127), dendritic cells (Cd209a, Flt3, Itgax encoding CD11c), monocytes (Ccr2, Ly6c2, Itgam encoding CD11b), granulocytes (Csf3r, S100a9), and CD4 +/CD8+ T cell subsets (Cd3e, Cd4, Cd8a). FIG. 9F Analysis of SWNT-positive (Cy5.5+) cells in each cluster confirms that SWNTs specifically target macrophages in the atherosclerotic aorta. Detection of SWNT uptake was greater in macrophages when characterizing cells by their whole-transcriptome, rather than the traditional limited markers used in flow cytometry above (FIG. 2,c and FIG. 8). ˜90% of lesional macrophages took up SWNTs in both SWNT-Cy5.5 and SWNT-SHP1i treated animals as compared to <30% of dendritic cells and <10% of T cells and granulocytes with SWNT detection. Similarly high 1 SWNT uptake (>75%) was detected in “macrophage-like” cells.

FIG. 10A-10J: FIG. 10A Survival analysis indicate no change in mortality with SWNT-SHP1i treatment, although SWNT-SHP1i-treated animals demonstrated a trend toward improved survival. FIG. 10B-10D The in vivo safety of pro-efferocytic SWNTs is further supported by the stable body weight in apoE^{-/-} mice treated with SWNT-SHP1i compared to SWNT-Cy5.5 controls. FIG. 10E No significant differences are observed in systolic blood pressure between treatment groups. FIG. 10AF-10G Similarly, there were no differences in the weight of any organ between groups. FIG. 10H-10J Hematology assessment demonstrate that SWNT-SHP1i treatment results in a significant decrease in the mean platelet volume (MPV) and platelet-large cell ratio (P-LCR). SWNT-SHP1i does not otherwise induce major hematopoietic toxicities, including a reduction in the red blood cell (RBC) count that is observed in anti-CD47 antibody treated mice FIG. 10H. There is also no effect of SWNT-SHP1i on total leukocytes FIG. 10I lipid levels, and clinical chemistry that would indicate organ toxicity or metabolic dysfunction FIG. 10J. Table of clinical chemistry and blood panels performed on mice intravenously injected with SWNT or SWNT-SHP1i. Note that procalcitonin (PCT) levels are also unchanged between treatment groups, indicating a low likelihood for increased bacterial infections in SWNT-SHP1i-treated mice. Data from anti-CD47 and IgG-treated mice in FIG. 10H have been reported. ⁸(Kojima, et al., 2016). *p<0.05 by unpaired two-tailed t-test.

FIG. 11: Large-scale phenome-wide association study (PheWAS) including nearly half a million individuals from the UK Biobank. PTPN6 variants are associated with alterations in the platelet index, platelet distribution width (p=3.90×10-9), and are not associated with other major adverse effects, such as anemia, respiratory infections (e.g. pneumonia), sepsis, and infectious causes of death.

FIG. 12.a, Assessment of serum stability of 89Zr-radiolabeled SWNTs demonstrates no signs of instability for up to 7 days at 37° C. in fresh mouse serum. Data are representative of 3independent experiments. b, Fluorescence-based studies show that the blood half-life (t_{1/2}) of SWNT-Cy5.5 measures ˜2 hr, indicating that desferrioxamine chelation of ⁸⁹Zr to SWNT-Cy5.5 does not significantly alter the circulation time of the nanoparticles used in the formal biodistribution studies (n=minimum 4 biologically independent animals per time point). Mean and s.e.m. are shown. c, Representation flow cytometry plots and gating strategy for analysis of SWNT uptake in homogenized organs. d, Immunofluorescence imaging shows SWNT (immunostained for PEG) accumulation in the aortic sinus, with lesser amounts in the spleen and liver, and little-to-no accumulation in other organs such as healthy aorta, lung, and kidney after 4 weeks of weekly serial injections. Data are representative of a minimum of 3 independent experiments. Scale bars, 100 μm.

FIG. 13.a, Representative flow cytometry plots from in vivo cellular uptake studies after 4 weeks of serial injections show significant SWNT accumulation in atherosclerotic Ly-6C^{hi} monocytes and macrophages, but low uptake by other vascular cells (n=4 biologically independent animals). b, Additional confocal images demonstrate co-localization (indicated by arrows) of SWNTs (green) with macrophages (red) in the atherosclerotic aortic sinus. Macrophages were identified by immunostaining for both CD68 (top) and Mac-3 (bottom). Data are representative of 4 independent experiments. Scale bars, 50 μm.

FIG. 14.a, Study timeline detailing the “angiotensin infusion” (which includes 4 weeks of high-fat diet and weekly SWNT injections) and “chronic atherosclerosis” models (which includes 2 weeks of high-fat diet, followed by 9 weeks of SWNT treatment without angiotensin II infusion). The beneficial effect of pro-efferocytic SWNT-SHP1i was confirmed in both models of vascular disease. b, In the main angiotensin infusion model, histological analysis of lesions in the aortic sinus area show that SWNT-SHP1i results in a significant reduction in plaque area in both male (n=9 biologically independent animals for control group, n=10 biologically independent animals for SWNT-SHP1i group) and female mice (n =8 biologically independent animals for control group, n=9 biologically independent animals for SWNT-SHP1i group), as measured by Oil Red O (ORO) staining. This finding is particularly important given the widely reported sex-dependent effects on atherosclerosis mouse models that is also relevant to human disease (Supplementary ref. 1). *p<0.05, **p<0.01 by unpaired two-tailed t-test. c, Similar therapeutic efficacy was observed in the chronic atherosclerosis models (n=12 biologically independent animals for control group, n=11 biologically independent animals for SWNT-SHP1i group). *p<0.05 by unpaired two-tailed t-test. Scale bar, 250 μm. d-f, The benefits of pro-efferocytic SWNT-SHP1i on atherosclerosis occur independently of blood pressure (n=6 biologically independent animals per group) (d), glucose (n=14 biologically independent animals for control group, n=17 biologically independent animals for SWNT-SHP1i group) (e), and cholesterol levels (n=11 biologically independent animals for control group, n=10 biologically independent animals for SWNT-SHP1i group) (f). Blue graphs indicate results from the angiotensin infusion model, while red graphs indicated results from the chronic atherosclerosis studies. For all graphs, data are expressed as the mean and s.e.m.

FIG. 15. Additional histological analyses confirm that SWNT-SHP1i induces a plaque-stabilizing phenotype. a, Masson Trichrome staining indicates that SWNT-SHP1i reduces the necrotic core in both male (n=8 biologically independent animals per group) and female mice (n=8 biologically independent animals per group). *p<0.05 by two-sided Mann-Whitney U test in left panel, by unpaired two-tailed t-test in right panel. Scale bar, 250 μm. b, A trend towards increased collagen content was also observed after treatment (n=8 biologically independent animals per group). c, Additional examples of lesion tracing and necrotic core analyses indicating reduced accumulation of apoptotic and necrotic debris after treatment. Data are representative of 16 independent experiments. Scale bar, 250 μm. d, α-SMA staining indicates enhanced smooth muscle cell content in the cap, suggesting a reduction in plaque vulnerability after therapy (n=8 biologically independent animals for control group, n=9 biologically independent animals for SWNT-SHP1i group). *p<0.05 by unpaired two-tailed t-test. Scale bar, 250 μm. e, Additional examples of lesional caspase staining (indicated with stars) highlighting a reduction in apoptotic cell content in treated animals. Data are representative of 9 independent experiments. Scale bar, 50μm. For all graphs, data are expressed as the mean and s.e.m.

FIG. 16. Schematic of the SWNT-TPI preparation process. Transmission electron microscopy (TEM) images, including by negative staining, show that PEG-functionalized SWNTs have a cylindrical appearance with a rigid diameter of 2-3 nm and PEG layer of 3-4 nm (FIG. 2a-b).

FIG. 17. Transmission electron microscopy of the nanodelivery vehicle and chemical characterization of DSPE-PEG modified SWNTs. a) TEM micrograph of SWNTs. White line-SWNT diameter is 2.2 nm. b) TEM micrograph of SWNTs with negative staining. Green dashes-core SWNT and red dashes-PEG layer. c) XPS spectra of raw SWNTs lacking a coating. d) XPS spectra of SWNT-DSPE-PEG.

FIG. 18. Chemical characterization of TPI-loaded SWNTs. a) UV-Vis spectroscopy of SWNT (no Cy5.5), SWNT, and SWNT-TPI; b) XPS analysis of SWNT; c) XPS analysis of SWNT-TPI; d) FT-IR spectroscopy of SWNTs, SWNT-TPI, and TPI.

FIG. 19. TPI release profiles from SWNT-TPI in a) saline buffers and b) serum (n=3/group, ***P<0.001). For all graphs, data are expressed as the mean and standard error of the mean (s.e.m.).

FIG. 20. High macrophage uptake upon exposure to SWNT-TPI, with no change in cell proliferation. a) The proliferation of RAW cells is not significantly affected by treatment with SWNT, SWNT-TPI, and TPI (ns: not significant; n=4 independent replicates, data are expressed as the mean +/− the standard error of the mean (s.e.m.). b) SWNT-TPI uptake by RAW macrophages in representative flow plots. SWNTs alone (upper right) and SWNT-TPI were incubated with RAW cells. All SWNT conditions displayed >99% uptake (n=4 independent replicates).

FIG. 21. SWNT-TPI drives macrophage efferocytosis. a) SWNT-TPI promotes the efferocytosis of apoptotic cells by macrophages RAW cells based on flow cytometry and b) SWNT-TPI significantly increases RAW cell phagocytic index (ns: not significant; *P=0.02, **P=0.005; n=4 independent replicates, data are expressed as the mean +/− the standard error of the mean (s.e.m.), each group is compared with PBS).

### DETAILED DESCRIPTION OF THE INVENTION

The present disclosure relates to methods of treating a subject for immune and/or inflammatory disorders involving defects in efferocytosis, including but not limited to atherosclerotic cardiovascular disease (ASCVD), atherosclerosis, and conditions such as CAD, peripheral artery disease (PAD) and cerebrovascular disease, by administering an agent that increases efferocytosis of apoptotic cells and cellular components such as those found in atherosclerotic plaques, including increasing efferocytosis of apoptotic smooth muscle cells. In some embodiments, the subject being treated with the disclosed compositions or in the methods of their use is homozygous or heterozygous for a 9p21 risk allele. In some embodiments, the agent that increases efferocytosis provides for one or more of the following activities: reactivates efferocytosis locally, increases efferocytosis in a tissue marked by a subset of monocytes in a diseased tissue, reduces vascular inflammation, reduces plaque burden, and/or inhibits the activity of the SHP1 phosphatase.

Coronary artery disease (CAD): is a narrowing or blockage of the arteries and vessels that provide oxygen and nutrients to the heart. It is caused by atherosclerosis, an accumulation of fatty materials on the inner linings of arteries. The resulting blockage restricts blood flow to the heart. When the blood flow is completely cut off, the result is a heart attack. CAD is the leading cause of death for both men and women in the United States.

Atherosclerosis (also referred to as arteriosclerosis, atheromatous vascular disease, arterial occlusive disease) as used herein, refers to a cardiovascular disease characterized by plaque accumulation on vessel walls and vascular inflammation. The plaque consists of accumulated intracellular and extracellular lipids, smooth muscle cells, connective tissue, inflammatory cells, and glycosaminoglycans. Inflammation occurs in combination with lipid accumulation in the vessel wall, and vascular inflammation is with the hallmark of atherosclerosis disease process.

Myocardial infarction is an ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in vessels previously partially obstructed by an atherosclerotic plaque enriched in inflammatory cells. Altered platelet function induced by endothelial dysfunction and vascular inflammation in the atherosclerotic plaque presumably contributes to thrombogenesis. Myocardial infarction can be classified into ST-elevation and non-ST elevation MI (also referred to as unstable angina). In both forms of myocardial infarction, there is myocardial necrosis. In ST-elevation myocardial infraction there is transmural myocardial injury which leads to ST-elevations on electrocardiogram. In non-ST elevation myocardial infarction, the injury is sub-endocardial and is not associated with ST segment elevation on electrocardiogram. Myocardial infarction (both ST and non-ST elevation) represents an unstable form of atherosclerotic cardiovascular disease. Acute coronary syndrome encompasses all forms of unstable coronary artery disease. Heart failure can occur as a result of myocardial dysfunction caused by myocardial infraction.

Angina refers to chest pain or discomfort resulting from inadequate blood flow to the heart. Angina can be a symptom of atherosclerotic cardiovascular disease. Angina may be classified as stable, which follows a regular chronic pattern of symptoms, unlike the unstable forms of atherosclerotic vascular disease. The pathophysiological basis of stable atherosclerotic cardiovascular disease is also complicated but is biologically distinct from the unstable form. Generally stable angina is not myocardial necrosis.

9p21 Risk. As used herein, the term “an individual carrying at least one 9p21 risk factor” refers to humans in which one or more risk alleles at the 9p21 locus are present in the genome. Such individuals have been shown to have an increased risk of: early onset myocardial infarction, abominal aortic aneurysm, stroke, peripheral artery disease, and myocardial infarction/coronary heart disease. This risk is independent of traditional risk factors, including diabetes, hypertension, cholesterol, and obesity. See, for example, Helgadottir et al. Science. 2007; 316(5830):1491-1493; Helgadottir et al. Nat Genet. 2008; 40(2):217-224; Palomaki et al. JAMA. 2010; 303(7):648-656; and Roberts et al. Curr Opin Cardiol. 2008; 23:629-633, each herein specifically incorporated by reference.

The 9p21 locus is in tight LD (linkage disequilibrium), and a number of single nucleotide polymorphisms (SNP) markers have been shown to be useful in diagnosis. Representative SN Ps include without limitation rs10757278; rs3217992; rs4977574; rs1333049; rs10757274; rs2383206; rs2383207; Rs3217989; rs1333040; rs2383207; rs10116277; rs7044859; rs1292136; rs7865618; rs1333045; rs9632884; rs10757272; rs4977574; rs2891168; rs6475606; rs1333048; rs1333049; Rs1333045; etc.

Efferocytosis. The process by which professional and nonprofessional phagocytes dispose of apoptotic cells in a rapid and efficient manner. Efferocytosis involves a number of molecules, including ligands on the apoptotic cells, e.g. phosphatidylserine; receptors on the efferocyte; soluble ligand-receptor bridging molecules; and so-called “find-me” and “don't-eat-me” molecules, e.g., lysosphospholipids and CD47, the expression of which by dying cells is altered to attract nearby phagocytes. By clearing apoptotic cells at a relatively early stage of cell death, when the cell plasma and organelle membranes are still intact, postapoptotic, or “secondary”, necrosis is prevented. Prevention of cellular necrosis, in turn, prevents the release of potentially damaging intracellular molecules into the extracellular milieu, including molecules that can stimulate inflammatory, proatherosclerotic and/or autoimmune responses.

The efficiency of efferocytic clearance in atherosclerotic lesions plays a key role in disease development. Efferocytosis is known to be impaired in human atherosclerotic plaque. A prominent feature of advanced atherosclerotic lesions is the necrotic core, or lipid core, which is a collection of dead and necrotic macrophages surrounded by inflammatory cells. Necrotic cores are thought to be a major feature responsible for plaque “vulnerability”, i.e., plaques capable of undergoing disruption and triggering acute lumenal thrombosis. Plaque disruption and acute thrombosis are the events that trigger acute coronary syndromes, including myocardial infarction, unstable angina, sudden cardiac death, and stroke.

By “manipulating efferocytosis” is meant an up-regulation or a down-regulation in efferocytosis of a targeted cell, e.g. apoptotic SMC, by about 10%, or about 20%, or 50%, or 70%, or 80% or 90%, or 100%, or 125%, or 150%, or 175% or about about 200% as compared to level of efferocytosis observed in absence of intervention.

The terms “phagocytic cells” and “phagocytes” are used interchangeably herein to refer to a cell that is capable of phagocytosis. There are three main categories of phagocytes: macrophages, mononuclear cells (histiocytes and monocytes); polymorphonuclear leukocytes (neutrophils) and dendritic cells. However, “non-professional” cells are also known to participate in efferocytosis, such as neighboring SMCs in the blood vessel wall.

“Treatment”, “treating”, “treat” and the like are used herein to generally refer to obtaining a desired pharmacologic and/or physiologic effect. The effect can be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. Those in need of treatment include individuals already diagnosed with CAD, e.g. atherosclerosis, as well as those in which the disease is to be prevented.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, rodents, domestic and farm animals, and zoo, sports, or pet animals, such as dogs, horses, cats, cows, sheep, goats, pigs, etc. Preferably, the mammal is human.

An “effective amount” is an amount sufficient to effect beneficial or desired clinical results. An effective amount can be administered in one or more administrations or doses. For purposes of the present disclosure, an effective amount of a therapeutic agent is an amount that is sufficient to palliate, ameliorate, stabilize, reverse, prevent, slow or delay the progression of the immune and/or inflammatory disease state, e.g. atherosclerosis or atherosclerotic plaque, by reversing the “don't eat me” anti-phagocytic signal and restoring efferocytosis in a target tissue or target cells. For example, in an animal model the percent of aortic surface area with atherosclerotic plaque may be reduced 25%, 50%, 75% or more relative to a control treated animal. Similar effects and comparable measurements indicating a reduction in the disease state may be obtained using such indicia appropriate for human patients, including without limitation C-reactive protein [CRP] and fibrinogen; lipoprotein-associated phospholipase A2 [Lp-PLA2] and myeloperoxidase [MPO]; growth differentiation factor-15 [GDF-15]) inflammatory markers; ambulatory arterial stiffness, IVUS imaging, and the like. See, for example Krintus et al. (2013) Crit Rev Clin Lab Sci. 11:1-17; Kollias et al. (2012) Atherosclerosis 224(2):291-301; and Kollias et al. (2011) Int. J. Cardiovasc. Imaging 27(2):225-37, each herein specifically incorporated by reference.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations. The definition also includes sample that have been enriched for particular types of molecules, e.g., nucleic acids, polypeptides, etc.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides, such as the high affinity binding of a SIRPα polypeptide to CD47; etc.) In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Suitable specific binding members include agents that specifically bind CD47, agents that specifically bind SIRPα, agents that otherwise block the interaction between CD47 and SIRPα, agents that bind to SHP1, and the like.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein. The variant polypeptides can have post-translational modifications not found on the natural protein.

A “fusion” polypeptide is a polypeptide comprising a polypeptide or portion (e.g., one or more domains) thereof fused or bonded to heterologous polypeptide. A fusion soluble CRT protein, for example, will share at least one biological property in common with a native sequence soluble CRT polypeptide. Examples of fusion polypeptides include immunoadhesins, as described above, which combine a portion of the polypeptide of interest with an immunoglobulin sequence, and epitope tagged polypeptides, which comprise a soluble polypeptide of interest or portion thereof fused to a “tag polypeptide”. The tag polypeptide has enough residues to provide an epitope against which an antibody can be made, yet is short enough such that it does not interfere with biological activity of the polypeptide of interest. Suitable tag polypeptides generally have at least six amino acid residues and usually between about 6-60 amino acid residues.

A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. The term “derivative” encompasses both amino acid sequence variants of polypeptide and covalent modifications thereof. For example, derivatives and fusion of soluble CRT find use as CRT mimetic molecules.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Incorporated by reference in its entirety, Patent Application Publication US 2014/0079630 discloses compositions and methods using SWNTs for molecular imaging for localizing tumors and visualizing target tissues. The application discloses the usefulness of SWNTs to precisely reach and enter tumors, and to provide chemotherapy to those tumors. Also described therein is the observation that SWNTs specifically enter a distinct set of monocytes in the mammalian subject's vasculature known as Ly-6C^{hi} monocytes in mice and CD14⁺ monocytes in human subjects, respectively. These monocytes act in a different manner than other monocytes in the blood vessels. Circulating SWNT-carrying monocytes move toward the blood vessel inner surface (endothelium), interacting with and moving along the surface; the SWNT-carrying monocytes and ultimately macrophages thereby infiltrate the tumor mass in murine tumor models. When the SWNTs, prior to being picked up by Ly-6C^{hi} monocytes or CD14+ monocytes, have been functionalized (conjugated to RGD peptides specific for the vasculature in the targeted tissue), the above described process of moving into diseased tissue, such as tumorous or atherosclerotic tissue, is notably accelerated. Thus, the RGD peptide-derivatized SWNTs were predicted to be useful for delivery of monoclonal antibodies or anti-tumor therapeutics for treating diseased tissues such as tumorous and atherosclerotic tissue.

Exemplary players in efferocytosis and/or the CD47-SIRPα pathway:

Cyclin-dependent kinase inhibitor 2 B (CDKN2B) is also known as multiple tumor suppressor 2 (MTS-2) or p15INK4B. The Genbank refseq for the human mRNA has the accession number NM_004936 and the protein refseq has the accession number NP_004927. This gene lies adjacent to the tumor suppressor gene CDKN2A in a region that is frequently mutated and deleted in a wide variety of tumors.

CDKN2B forms a complex with CDK4 or CDK6, and prevents the activation of the CDK kinases by cyclin D; thus the encoded protein functions as a cell growth regulator that inhibits cell cycle G1 progression. It was previously reported that decreased CDKN2B expression associated with 9p21 risk alleles impairs expression of calreticulin, a ligand required for activation of engulfment receptors on phagocytic cells. As a result, cdkn2B-deficient apoptotic bodies, e.g. apoptotic smooth muscle cells, are rendered resistant to efferocytosis and are not efficiently cleared by phagocytic cells. Agents that activate or upregulate CDKN2B expression are known in the art, including, for example, palbociclib (see Toogood et al. (2005) J Med Chem. 48(7):2388-406); 5-aza-2′-deoxycytidine in the absence or presence of phenylbutyrate (see Lemaire et al. (2004) Leuk Lymphoma. 45(1):147-54); hypoxia-inducible-factors-1a and -2a (see Aesoey et al. (2013) Endocr Rev, Vol. 34 (03_Meeting Abstracts): SUN-303; etc. Agents that activate or upregulate CDKN2B also can be determined by screening methods as known in the art.

Calreticulin. Calreticulin is a multifunctional protein of 417 amino acids, molecular weight 48kDa, that binds Ca²⁺ ions, rendering it inactive. The Ca²⁺ is bound with low affinity, but high capacity, and can be released on a signal. Calreticulin can be located in storage compartments associated with the endoplasmic reticulum, where it binds to misfolded proteins and prevents them from being exported to the Golgi apparatus. Calreticulin is also found in the nucleus, suggesting that it may have a role in transcription regulation. Calreticulin binds to the synthetic peptide KLGFFKR, which is almost identical to an amino acid sequence in the DNA-binding domain of the superfamily of nuclear receptors. The gene symbol for calreticulin is CALR, and the human sequences may be accessed at Pubmed as follows: Protein Accession# NP_004334; Nucleotide Accession#: NM_004343.

Calreticulin on the surface of apoptotic cells serves as a recognition and clearance ligand by activating the internalization receptor LRP on the responding phagocyte cell surface. The surface expression of calreticulin increases and calreticulin was redistributed during apoptosis, possibly enhancing stimulation of LRP on the phagocyte.

The low density lipoprotein receptor-related protein (LRP) is a 4,544-amino acid protein containing a single transmembrane segment, with a high degree of sequence identity to the LDL receptor. The human genetic sequences may be accessed at Pubmed as follows: Nucleotide Accession#: NM_002332.2 GI:126012561.

Agents that specifically bind to calreticulin (CRT) are of interest as agonists for enhancing the pro-phagocytic activity of CRT. CRT binding agents useful in the methods of the invention include analogs, derivatives and fragments of the original specific binding member, e.g. Fab fragments of antibodies, etc. Calreticulin “mimetics” and “agonists” include molecules that function similarly to or potentiate CRT by binding and activating LRP receptor. Molecules useful as CRT mimetics include derivatives, variants, and biologically active fragments of naturally occurring CRT. Molecules useful as agonists include antibodies and other agents that act to enhance the pro-phagocytic activity of CRT.

Fragments of soluble CRT, particularly biologically active fragments and/or fragments corresponding to functional domains, are of interest. Fragments of interest will typically be at least about 10 aa to at least about 15 aa in length, usually at least about 50 aa in length, but will usually not exceed about 142 aa in length, where the fragment will have a stretch of amino acids that is identical to CRT. A fragment “at least 20 aa in length,” for example, is intended to include 20 or more contiguous amino acids from, for example, the polypeptide encoded by a cDNA for CRT. In this context “about” includes the particularly recited value or a value larger or smaller by several (5, 4, 3, 2, or 1) amino acids.

In vitro assays for calreticulin biological activity include, e.g. phagocytosis of porcine cells by human macrophages, binding to LRP, etc. A candidate agent useful as a calreticulin agonist mimetic results in the down regulation of phagocytosis by at least about 10%, at least about 20%, at least about 50%, at least about 70%, at least about 80%, or up to about 90% compared to level of phagocytosis observed in absence of candidate agent.

CD47, also known as integrin associated protein (IAP,) is a 50 kDa membrane receptor that has extracellular N-terminal IgV domain, five transmembrane domains, and a short C-terminal intracellular tail transmembrane, belonging to the immunoglobulin superfamily, with interacts with integrins, most commonly integrin αvβ3, thrombospondin-1 (TSP-1) and signal-regulatory protein alpha (SIRPα. The reference sequence for the human mRNA has the Genbank accession number NM_001025079, and the protein reference sequence is NP_001768.

The CD47/SIRPα interaction leads to bidirectional signaling, resulting in different cell-to-cell responses including inhibition of phagocytosis, stimulation of cell-cell fusion, and T-cell activation

As used herein, the term “anti-CD47 agent” refers to any agent that reduces the binding of CD47 (e.g., on an affected cell) to SIRPα (e.g., on a phagocytic cell). In some embodiments the anti-CD47 agent does not interfere or bind to the regions of CD47 that bind to thrombospondin. In some embodiments, the anti-CD47 agent does not activate CD47 upon binding. When CD47 is activated, a process akin to apoptosis (i.e., programmed cell death) occurs (Manna and Frazier, Cancer Research, 64, 1026-1036, Feb. 1 2004). Thus, in some embodiments, the anti-CD47 agent does not directly induce cell death of a CD47-expressing cell.

Non-limiting examples of suitable anti-CD47 reagents include high affinity SIRPα reagents, anti-SIRPα antibodies, and anti-CD47 antibodies or antibody fragments. In some embodiments, a suitable anti-CD47 agent (e.g. an anti-CD47 antibody, a high affinity SIRPα reagent, etc.) specifically binds CD47 to reduce the binding of CD47 to SIRPα. In some embodiments, a suitable anti-CD47 agent (e.g., an anti-SIRPα antibody, etc.) specifically binds SIRPα to reduce the binding of CD47 to SIRPαA suitable anti-CD47 agent that binds SIRPα does not activate SIRPα (e.g., in the SIRPα-expressing phagocytic cell).

The efficacy of a suitable anti-CD47 agent can be assessed by assaying the agent. An agent for use in the methods of the invention will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 160%, or at least 200%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In one embodiment of the invention, the anti-CD47 agent, or a pharmaceutical composition comprising the agent, is provided in an amount effective to detectably inhibit the binding of CD47 to SIRPα present on the surface of phagocytic cells. The effective amount is determined via empirical testing routine in the art, for example in a biological sample taken from an infected individual. The effective amount may vary depending on the number of cells being targeted, the location of the cells, and factors specific to the subject.

High affinity SIRPα reagent. In some embodiments, a subject anti-CD47 agent is a “high affinity SIRPα reagent”, which includes SIRPα -derived polypeptides and analogs thereof. High affinity SIRPα reagents are described in international application PCT/US13/21937, which is hereby specifically incorporated by reference. High affinity SIRPα reagents are variants of the native SIRPα protein. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

A high affinity SIRPα reagent comprises the portion of SIRPα that is sufficient to bind CD47 at a recognizable affinity, e.g., high affinity, which normally lies between the signal sequence and the transmembrane domain, or a fragment thereof that retains the binding activity. The high affinity SIRPα reagent will usually comprise at least the d1 domain of SIRPα with modified amino acid residues to increase affinity. In some embodiments, a SIRPα variant of the present invention is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. In other embodiments, the second polypeptide is any suitable polypeptide that is substantially similar to Fc, e.g., providing increased size, multimerization domains, and/or additional binding or interaction with Ig molecules.

A suitable high affinity SIRPα reagent reduces (e.g., blocks, prevents, etc.) the interaction between the native proteins SIRPα and CD47. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, etc.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Non-limiting examples of suitable antibodies include clones B6H12, 5F9, 8B6, and C3 (for example as described in International Patent Publication WO 2011/143624, herein specifically incorporated by reference).

Anti-SIRPα antibodies. In some embodiments, a subject anti-CD47 agent is an antibody that specifically binds SIRPα (i.e., an anti-SIRPα antibody) and reduces the interaction between CD47 on one cell (e.g., an infected cell) and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of infected cells over non-infected cells. Those cells that express higher levels of CD47 (e.g., infected cells) relative to other cells (non-infected cells) will be preferentially phagocytosed. Thus, a suitable anti-SIRPα antibody specifically binds SIRPα without activating/stimulating enough of a signaling response to inhibit phagocytosis.

As used herein, antibodies used in the presently described methods include fully human, humanized or chimeric versions of such antibodies. Humanized antibodies are especially useful for in vivo applications in humans due to their low antigenicity. Similarly caninized, felinized, etc antibodies are especially useful for applications in dogs, cats, and other species respectively.

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Exemplary small molecules used in the presently disclosed compositions and methods to inhibit the SHP1 PTPase were: TPI-1 and NSC-87877. TPI-1 (CAS 79756-69-7) inhibits SHP1. The SHP1/2 PTPase inhibitor NSC-87877 (CAS 56932-43-5, Calbiochem) is a cell-permeable, potent, catalytic site-targeting inhibitor of both the SHP-1 and SHP-2 tyrosine phosphatases.

Pro-efferocytic nanoparticles are specifically taken up by lesional macrophages and prevent atherosclerosis

Monocytes are circulating blood cells that constitute approximately 10% of peripheral leukocytes (white blood cells) in humans (Yona et al., 2009). One of the subsets of monocytes are Ly-6C^{hi} monocytes. Monocytes develop in the bone marrow, and upon infection, a large number of Ly-6C^{hi} monocytes exit the bone marrow into the peripheral circulation. In fact, it appears that the total number of Ly-6C^{hi} monocytes increases upon infection. They naturally migrate to sites of inflammation, where the Ly-6C^{hi} monocytes can develop into macrophages and dendritic cells. It has also been found that in the absence of inflammation, the number of Ly-6C^{hi} monocytes in the peripheral blood decreases significantly. Thus, Ly-6C^{hi} monocytes naturally move toward inflamed tissue. In some embodiments of the present disclosure, this innate homing has been built upon to provide a system for targeted delivery of a therapeutic agent to diseased tissues having dysregulated efferocytosis in immune and/or inflammatory diseases, e.g. atherosclerotic tissue, and to provide therapies that treat the diseased tissue.

Diseased and dying cells within atherosclerotic plaques are ideally meant to be swiftly and efficiently cleared out via a phagocytic process known as efferocytosis to prevent the inflammatory consequences associated with the accumulation of apoptotic debris. However, dysregulation of efferocytosis can lead to the enlargement of dangerous plaques that cause heart attacks, strokes and other cardiovascular complications. Cell-surface expression of anti-phagocytic “don't eat me” signals such as expression of CD47 help regulate efferocytosis. It was recently found that CD47, a key anti-phagocytic molecule, is upregulated significantly in atherosclerotic cardiovascular disease (as in many cancers), the leading cause of death worldwide. This finding raised the possibility of pro-efferocytic therapies by blocking the CD47 signal and treating atherosclerosis. Yet the anti-CD47 antibodies currently used for therapy in animal models have side effects (off-target effects), such as anemia.

As a solution to this challenge, the presently disclosed compositions and methods provide a targeted delivery system that is highly selective to direct a therapeutic agent to monocytes/macrophages based on the use of single-walled carbon nanotubes (SWNTs), which are almost exclusively taken up by a single immune cell subset, Ly-6C^{hi} monocytes. These monocytes naturally home to atherosclerotic plaques in mice, with a similar process thought to occur in humans as well. Instead of using CD47 antibodies, the same CD47-SIRPα pathway was inhibited by employing a small molecule inhibitor of a downstream tyrosine phosphatase enzyme, SHP1. These inhibitors were linked to the selective SWNT technology (SHP1 inhibitor is bound via pi-pi interactions to the SWNT surface, where the SWNT is coated with amphiphilic PEG and Cy5.5 dye). These selective SWNTs thus specifically deliver the SHP1 inhibitor to the monocytes/macrophages that perform efferocytosis, reactivating them without side effects, in an advanced immune-based delivery strategy. Thus, SWNTs can selectively deliver a small molecule inhibitor of the SHP1 tyrosine phosphatase enzyme downstream of the CD47-SIRPα anti-efferocytic pathway as described herein to diseased tissues marked by the presence of Ly-6C^{hi} monocytes.

As noted above, atherosclerosis, the process underlying heart attack and stroke, is the world's leading killer. A characteristic feature of the atherosclerotic plaque is the accumulation of apoptotic cells in the necrotic core. Therapies that stimulate the phagocytic clearance of these cells represent an emerging approach to directly combat atherosclerosis. However, the systemic, pro-phagocytic antibody-based therapies currently in development can cause the off-target clearance of healthy tissues, leading to toxicities such as anemia. Here, a highly macrophage-selective nanotherapy was developed, comprised by single-walled carbon nanotubes (SWNTs) that have been loaded with a chemical inhibitor of the anti-phagocytic CD47-SIRPα signaling axis. These SWNTs were demonstrated to specifically home to the atherosclerotic plaque, reactivate lesional phagocytosis, and reduce plaque burden in atheroprone apoE^{-/-} mice without compromising safety, thereby overcoming a key translational barrier for this class of drugs. Single-cell RNA sequencing analysis reveals that pro-phagocytic SWNTs decrease the expression of inflammatory genes linked to cytokine and chemokine pathways in lesional macrophages. Together, these data demonstrate the potential of Trojan horse nanotubes as a platform for the prevention of atherosclerotic cardiovascular disease.

The presently described targeted drug delivery system can thus remarkably reduce the required dosage of the drug, due to selectively deliver the drug to the target area, and minimize drug-associated side effects. Moreover, since lower dosage of drug is needed and no antibody is required, it is likely to be a more cost effective solution.

Applications:

The inhibitor-loaded SWNTs can be used as a targeted, minimally-invasive monocyte-selective immunotherapy to treat or prevent atherosclerosis (which otherwise lead to heart attacks, strokes and other cardiovascular events).

Advantages & improvements over existing methods, devices or materials:

The key advantage of this therapy over anti-CD47-based treatments is that the therapeutic agent is selectively delivered, leading to significantly reduced side effects. Additionally, compared to stents and surgical treatments, the present approach is non-invasive and likely to be as or more efficacious.

SWNTS 1) loaded with both small molecules and antibodies (CD47 antibodies) and 2) labeled with fluorescent dyes were synthesized and successfully characterized. In vitro cell experiments suggest greater efficacy for this system (i.e., in improving efferocytosis) compared with the former standard (anti-CD47 treatment) and initial in vivo experiments in an atherosclerosis mouse model successfully reduce atherosclerosis based on an Oil Red O (ORO) analysis, confirming the great potential of this system for targeted treatment of atherosclerosis. In vivo data proving the safety and efficacy of this therapy is available.

The following experiments use the materials and methods described below.

### Methods

In the present disclosure, methods are provided for treating or reducing atherosclerosis using a system involving SWNTs for introducing a therapeutic agent to a target tissue or cells in an individual, for restoring and/or increasing efferocytosis of apoptotic cells (e.g., smooth muscle cells) or cellular degradation components in diseased tissues (e.g., coronary or extracardiac plaques). In some embodiments, the individual is homozygous or heterozygous for a 9p21 risk allele. In some embodiments, the therapeutic agent that increases efferocytosis provides for one or more of the following activities: inhibiting a component of the CD47-SIRPα anti-phagocytic pathway (e.g., SHP1), reducing the binding of CD47 to SIRPα; increasing or mimicking the activity of calreticulin, including binding of calreticulin to LRP; or increasing expression of CDKN2B. Such methods include administering to a subject in need of treatment a therapeutically effective amount or an effective dose of a composition comprising SWNTs and an efferocytosis stimulating agent, including without limitation combinations of the agent with another drug that treats an immune and/or inflammatory disease or disorder. Methods of administration to the cardiovascular/circulatory system are of interest, although oral formulations may also find use.

Effective doses of the therapeutic entity of the present disclosure vary depending upon many different factors, including the nature of the agent, means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage can range from about 0.0001 to 500 mg/kg, and more usually 0.01 to 100 mg/kg, of the host body weight. For example dosages can be 1mg/kg body weight or 10 mg/kg body weight or within the range of 1-50 mg/kg. The dosage may be adjusted for the molecular weight of the reagent. An exemplary treatment regime entails administration daily, semi-weekly, weekly, once every two weeks, once a month, etc. In another example, treatment can be given as a continuous infusion. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient. It will be understood by one of skill in the art that such guidelines will be adjusted for the molecular weight of the active agent, e.g. in the use of polypeptide fragments, in the use of antibody conjugates, in the use of high affinity SIRPα reagents, etc. The dosage may also be varied for localized administration, e.g. intranasal, inhalation, etc., or for systemic administration, e.g. i.m., i.p., i.v., and the like.

For the treatment of disease, the appropriate dosage of the agent will depend on the severity and course of the disease, whether the agent is administered for preventive purposes, previous therapy, the patients clinical history and response to the antibody, and the discretion of the attending physician. The agent is suitably administered to the patient at one time or over a series of treatments.

Suitable agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an efferocytosis stimulating agent includes use in combination with another therapeutic agent, e.g., drugs useful in the treatment of atherosclerosis. Such combinations may include, without limitation, statins. Statins are inhibitors of HMG-CoA reductase enzyme. These agents are described in detail; for example, mevastatin and related compounds as disclosed in U.S. Pat. No. 3,983,140; lovastatin (mevinolin) and related compounds as disclosed in U.S. Pat. No. 4,231,938; pravastatin and related compounds as disclosed in U.S. Pat. No. 4,346,227; simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat Nos. 4,681,893, 5,273,995 and 5,969,156; and cerivastatin and related compounds as disclosed in U.S. Pat. Nos. 5,006,530 and 5,177,080. Additional agents and compounds are disclosed in U.S. Pat. Nos. 5,208,258, 5,130,306, 5,116,870, 5,049,696, RE 36,481, and RE 36,520. Statins include the salts and/or ester thereof.

Other drugs useful in combination include, for example, fibrates such as gemfibrozil, fenofibrate, etc.; niacin; zetia; bile acid sequestrants, e.g. cholestyramine, colestipol, colesevelam; lovaza, vascepa; drugs to reduce hypertension, etc.

Therapeutic formulations comprising one or more agents of the invention are prepared for storage by mixing the agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the agent to be administered will be governed by such considerations, and is the minimum amount necessary to treat or prevent atherosclerosis.

The agent can be administered by any suitable means, including topical, oral, parenteral, subcutaneous, intraperitoneal, intrapulmonary, and intranasal. Parenteral infusions include intramuscular, intravenous, intraarterial, intraperitoneal, intrathecal or subcutaneous administration. In addition, the agent can be suitably administered by pulse infusion, particularly with declining doses of the agent.

The agent need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

An agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an anti-CD47 agent by non-covalent associations, such as non-covalent bonding or by encapsulation. The nature of the carrier can be either soluble or insoluble for purposes of the invention. Those skilled in the art will know of other suitable carriers for binding anti-CD47 agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the anti-CD47 agent through an acceptable chemical linker or chelation carrier. Positron emitting moieties for use in the present invention include ¹⁸F, which can be easily conjugated by a fluorination reaction with the agent.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

### Genetic Screening

In one aspect of the present invention, an individual is tested for the presence of a 9p21 risk allele prior to treatment. Such methods comprise an analysis of genomic DNA in an individual for a 9p21 allele that confers an increased susceptibility to atherosclerosis. Individuals are screened by analyzing their genomic sequence at 9p21, e.g. rs10757278 or rs1333049 or another representative 9p21 SNP sequences for the presence of a predisposing allele, as compared to a normal sequence.

A number of methods are used for determining the presence of a predisposing variant in an individual. Genomic DNA is isolated from the individual or individuals that are to be tested. DNA can be isolated from any nucleated cellular source such as blood, hair shafts, saliva, mucous, biopsy, feces, etc. Methods using PCR amplification can be performed on the DNA from a single cell, although it is convenient to use at least about 10⁵ cells. Where large amounts of DNA are available, the genomic DNA is used directly. Alternatively, the region of interest is cloned into a suitable vector and grown in sufficient quantity for analysis, or amplified by conventional techniques. Of particular interest is the use of the polymerase chain reaction (PCR) to amplify the DNA that lies between two specific primers. The use of the polymerase chain reaction is described in Saiki et al. (1985) Science 239:487, and a review of current techniques may be found in McPherson et al. (2000) PCR (Basics: From Background to Bench) Springer Verlag; ISBN: 0387916008. A detectable label may be included in the amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Primer pairs are selected from the genomic sequence using conventional criteria for selection. The primers in a pair will hybridize to opposite strands, and will collectively flank the region of interest. The primers will hybridize to the complementary sequence under stringent conditions, and will generally be at least about 16 nt in length, and may be 20, 25 or 30 nucleotides in length. The primers will be selected to amplify the specific region suspected of containing the predisposing mutation. Typically the length of the amplified fragment will be selected so as to allow discrimination between repeats of 3to 7 units. Multiplex amplification may be performed in which several sets of primers are combined in the same reaction tube, in order to analyze multiple exons simultaneously. Each primer may be conjugated to a different label.

The exact composition of the primer sequences are not critical to the invention, but they must hybridize to the flanking sequences under stringent conditions. Criteria for selection of amplification primers are as previously discussed. To maximize the resolution of size differences at the locus, it is preferable to choose a primer sequence that is close to the SNP sequence, such that the total amplification product is at least about 30, more usually at least about 50, preferably at least about 100 or 200 nucleotides in length, which will vary with the number of repeats that are present, to not more than about 500 nucleotides in length. The number of repeats has been found to be polymorphic, as previously described, thereby generating individual differences in the length of DNA that lies between the amplification primers. Conveniently, a detectable label is included in the amplification reaction. Multiplex amplification may be performed in which several sets of primers are combined in the same reaction tube. This is particularly advantageous when limited amounts of sample DNA are available for analysis. Conveniently, each of the sets of primers is labeled with a different fluorochrome.

After amplification, the products can be size fractionated. Fractionation may be performed by gel electrophoresis, particularly denaturing acrylamide or agarose gels. A convenient system uses denaturing polyacrylamide gels in combination with an automated DNA sequencer, see Hunkapillar et al. (1991) Science 254:59-74. The automated sequencer is particularly useful with multiplex amplification or pooled products of separate PCR reactions. Capillary electrophoresis may also be used for fractionation. A review of capillary electrophoresis may be found in Landers, et al. (1993) BioTechniques 14:98-111. The size of the amplification product is proportional to the number of repeats (n) that are present at the locus specified by the primers. The size will be polymorphic in the population, and is therefore an allelic marker for that locus. The amplified or cloned fragment is alternatively sequenced by various high methods known in the art.

The presence of a predisposing risk allele is indicative that an individual is at increased risk of developing atherosclerosis and may benefit from treatment by the methods of the invention, although the methods can additionally find use in individuals without a 9p21 genetic risk factor. The diagnosis of a disease predisposition allows the affected individual to seek early treatment of potential lesions, and to avoid activities that increase risk for cardiovascular disease.

### Drug Screening

Screening assays identify compounds that modulate the expression or activity of proteins involved in efferocytosis, including without limitation CDKN2B, calreticulin, CD47, SIRPα, SHP1, etc. An efferocytosis stimulating agent can, for example, act as the basis for amelioration of such cardiovascular diseases as atherosclerosis, ischemia/reperfusion, hypertension, restenosis, and arterial inflammation. Such compounds may include, but are not limited to peptides, antibodies, or small organic or inorganic compounds. Methods for the identification of such compounds are described below.

Cell- and animal-based systems can act as models for cardiovascular disease and are useful in such drug screening. The animal- and cell-based models may be used to identify drugs, pharmaceuticals, therapies and interventions that are effective in treating cardiovascular disease. In addition, such animal models may be used to determine the LD₅₀ and the ED₅₀ in animal subjects, and such data can be used to determine the in vivo efficacy of potential cardiovascular disease treatments. Animal-based model systems of cardiovascular disease may include, but are not limited to, non-recombinant and engineered transgenic animals. Non-recombinant, non-genetic animal models of atherosclerosis may include, for example, pig, rabbit, or rat models in which the animal has been exposed to either chemical wounding through dietary supplementation of LDL, or mechanical wounding through balloon catheter angioplasty, for example. Additionally, animal models exhibiting cardiovascular disease symptoms may be engineered by utilizing, for example, smooth muscle cell marking, knockouts of CDKN2B, etc. gene sequences in conjunction with techniques for producing transgenic animals that are well known to those of skill in the art. For example, target gene sequences may be introduced into, and knocked out or overexpressed in the genome of the animal of interest. Animals of any species, including, but not limited to, mice, rats, rabbits, guinea pigs, pigs, micro-pigs, goats, and non-human primates, e.g., baboons, monkeys, and chimpanzees may be used to generate cardiovascular disease animal models.

Any technique known in the art may be used to introduce a target gene transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc.

Specific cell types within the animals may be analyzed and assayed for cellular phenotypes characteristic of cardiovascular disease. In the case of monocytes, such phenotypes may include but are not limited to increases in rates of LDL uptake, adhesion to endothelial cells, transmigration, foam cell formation, fatty streak formation, and production of foam cell specific products. Further, such cellular phenotypes may include a particular cell type's fingerprint pattern of expression as compared to known fingerprint expression profiles of the particular cell type in animals exhibiting cardiovascular disease symptoms. The ability of smooth muscle cells to be taken up by phagocytes is of particular interest.

Cells that are down-regulated in CDKN2B activity can be utilized to identify compounds that exhibit anti-cardiovascular disease activity. In the case of monocytes, such phenotypes may include but are not limited to increases in rates of LDL uptake, adhesion to endothelial cells, transmigration, foam cell formation, fatty streak formation, and production by foam cells of growth factors such as bFGF, IGF-I, VEGF, IL-1, M-CSF, TGFβ, TGFα, TNFα, HB-EGF, PDGF, IFN-γ and GM-CSF. Transmigration rates, for example, may be measured using an in vitro system to quantify the number of monocytes that migrate across the endothelial monolayer and into the collagen layer of the subendothelial space.

In vitro systems may be designed to identify compounds capable of activating efferocytosis. Such compounds may include, but are not limited to, peptides made of D-and/or L-configuration amino acids, phosphopeptides, antibodies, and small organic or inorganic molecules. The principle of the assays used to identify compounds that upregulate CDKN2B or calreticulin involves preparing a reaction mixture of the protein and a test compound under conditions and for a time sufficient to allow the two components to interact, and detecting the resulting change in the desired biological activity. Alternatively, a simple binding assay can be used as an initial screening method. These assays can be conducted in a variety of ways. For example, one method to conduct such an assay would involve anchoring a protein or a test substance onto a solid phase and detecting complexes anchored on the solid phase at the end of the reaction.

In a binding assay, the reaction can be performed on a solid phase or in liquid phase. In a solid phase assay, the nonimmobilized component is added to the coated surface containing the anchored component. After the reaction is complete, unreacted components are removed under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a binding reaction can be conducted in a liquid phase, the reaction products separated from unreacted components, and complexes detected; e.g., using an immobilized antibody specific for target gene product or the test compound to anchor any complexes formed in solution, and a labeled antibody specific for the other component of the possible complex to detect anchored complexes.

Cell-based systems such as those described above may be used to identify compounds that act to ameliorate cardiovascular disease symptoms. For example, such cell systems may be exposed to a test compound at a sufficient concentration and for a time sufficient to elicit such an amelioration of cardiovascular disease symptoms in the exposed cells. After exposure, the cells are examined to determine whether one or more of the cardiovascular disease cellular phenotypes has been altered to resemble a more normal or more wild type, non-cardiovascular disease phenotype.

In addition, animal-based disease systems, such as those described, above may be used to identify compounds capable of ameliorating disease symptoms. Such animal models may be used as test substrates for the identification of drugs, pharmaceuticals, therapies, and interventions, which may be effective in treating disease. For example, animal models may be exposed to a compound, suspected of exhibiting an ability to ameliorate cardiovascular disease symptoms, at a sufficient concentration and for a time sufficient to elicit such an amelioration of disease symptoms in the exposed animals. The response of the animals to the exposure may be monitored by assessing the reversal of disorders associated with disease, for example, by counting the number of atherosclerotic plaques and/or measuring their size before and after treatment.

With regard to intervention, any treatments that reverse any aspect of cardiovascular disease symptoms should be considered as candidates for human disease therapeutic intervention. Dosages of test agents may be determined by deriving dose-response curves.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The invention now being fully described, it will be apparent to one of ordinary skill in the art that various changes and modifications can be made without departing from the spirit or scope of the invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The phagocytic clearance of apoptotic cells is a routine component of tissue homeostasis, protecting tissue from exposure to the inflammatory contents of dying cells.¹⁻³ To remove these cells, the body engages in a process known as efferocytosis, from the Latin word meaning “to take to the grave”. Efferocytosis is a highly conserved process triggered by “eat me” ligands which signal to phagocytes to induce engulfment.1 Conversely, cells may overexpress “don't eat me” ligands to avoid removal.⁴

By delivering an anti-phagocytic signal that enables immune evasion, the upregulation of the “don't eat me” molecule, CD47, is a major mechanism by which a variety of cancers establish and propagate disease. Most recently, CD47 signaling was discovered to play a significant role in atherosclerosis. Atherosclerosis, the process underlying heart attack and stroke, has remained the leading cause of death in the United States for nearly the past century. While pursuing the mechanism by which apoptotic vascular cells escape clearance from the diseased artery, it was discovered that CD47 is markedly upregulated in atherosclerotic plaques.

CD47 functions as a ligand for the signal regulatory protein-α (SIRPαon macrophages. Following this interaction, SIRPα activates the SH2 domain-containing phosphatase-1 (SHP-1) to mediate the intracellular signaling that suppresses phagocytic function. This signaling cascade renders diseased vascular cells resistant to removal and promotes plaque expansion. In hyperlipidemic mice, CD47-blocking antibodies (Ab) normalize the defect in efferocytosis, prevent the progression of established lesions, and protect against plaque rupture. However, antibody-mediated blockade of CD47 also accelerates the off-target removal of certain healthy tissue, most notably eliminating red blood cells (RBCs) in the spleen. The resulting anemia and reduced oxygen-carrying capacity may exacerbate ischemia in individuals with atherosclerotic disease, thus limiting the translational potential of systemic, pro-efferocytic therapies currently in development.

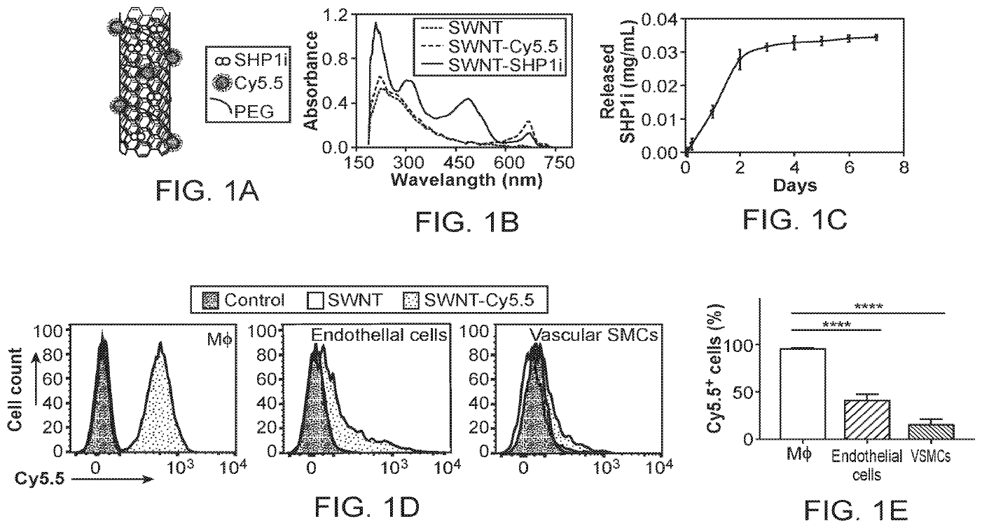

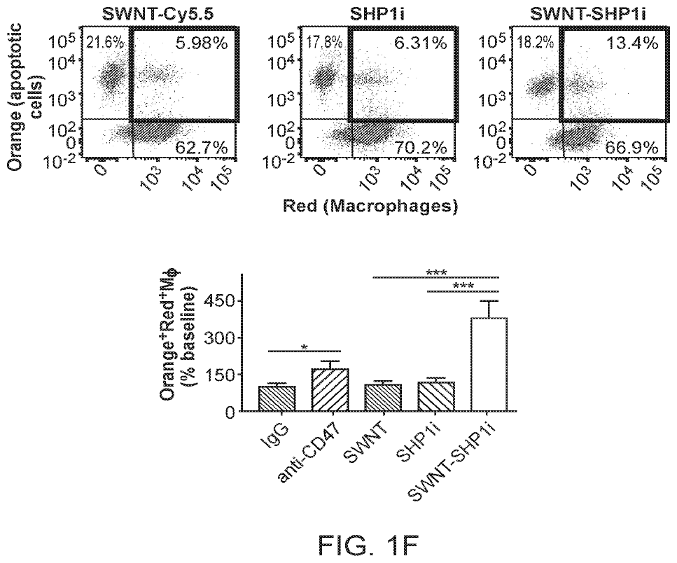

To develop a method that more specifically and safely restores impaired efferocytic activity, nanoparticles (NPs) were precision-engineered for targeted delivery of therapeutic agents and shown to interrupt CD47-SIRPα signaling in monocytes and macrophages. The system, termed SWNT-SHP1i, involves a backbone of polyethylene glycol (PEG)-functionalized single-walled carbon nanotubes (SWNTs) loaded with (1) a fluorescent probe Cy5.5 and (2) a small-molecule inhibitor of CD47′s downstream effector molecule, SHP-1 (FIG. 1,a). PEG-functionalized SWNTs were chosen because of their ultrahigh loading capacity, favorable toxicology, and the recent demonstration of their selectivity for a specific leukocyte subset, Ly-6C^{hi} monocytes (inflammatory monocytes). The selectivity for this cell type is important, as Ly-6C^{hi} monocytes are the primary circulating cells recruited to the diseased artery, where they differentiate into lesional macrophages. In addition to regulating the inflammatory response, macrophages have a homeostatic role as phagocytes that scavenge lipids and apoptotic debris. Because their phagocytic capacity becomes impaired in advanced atherosclerosis, strategies which restore the “appetite” of macrophages for uncleared apoptotic cells have the potential to both combat plaque expansion and prevent the inflammation which results from post-apoptotic necrosis. Leveraging SWNTs as a “Trojan horse” would enable plaque-specific modulation of the CD47-SIRPα-SHP1 axis, thereby promoting the clearance of diseased cells in the lesion, while minimizing toxicities elsewhere in the body.

### Example 1

### Preparation and Characterization of SWNT-SHP1i

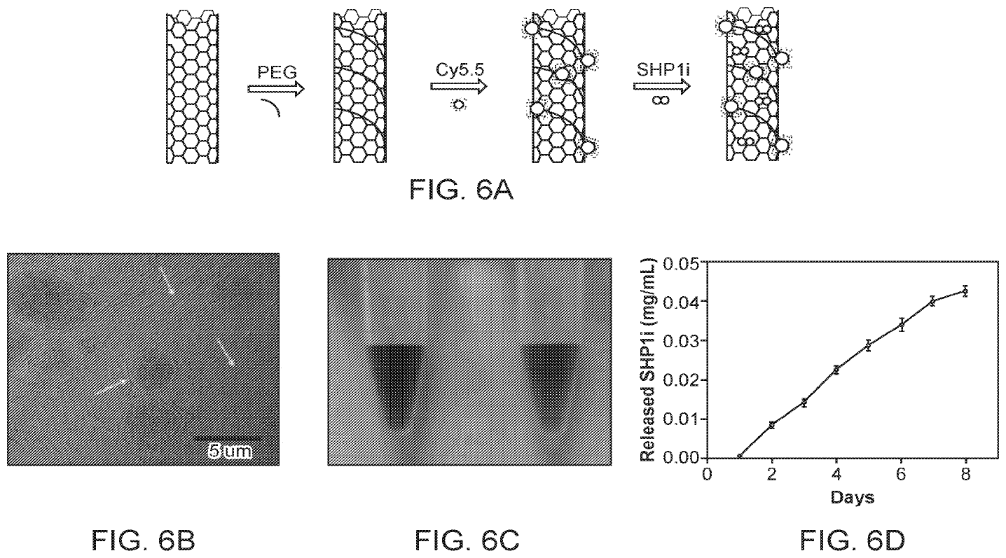

After fabricating SWNT-PEG-Cy5.5 (SWNT-Cy5.5) as previously described, they were loaded with a SHP1 inhibitor (SHP1i) (FIG. 1,a and FIG. 6). PEG was used to solubilize SWNTs in aqueous solutions, endow biocompatibility, and prolong in vivo circulation times. Fluorescent Cy5.5 dye was used for flow cytometric characterization and loaded SHP1i onto SWNTs through π-π stacking and hydrophobic interactions. UV-visible spectroscopy validated the presence of Cy5.5 (sharp peak at 674 nm) and SHP1i loading (absorption peaks at 230, 320, 490 nm over the characteristic SWNT absorption spectrum) (FIG. 1,b). The presence of Cy5.5 and SHP1i on SWNTs 1 was further confirmed by 1.) the visible color change in the SWNT-SHP1i solution upon SHP1i adsorption; 2.) attenuated total reflectance (ATR) infrared spectroscopy (FIGS. 6); and 3) the shift in λ-potential of SWNT-Cy5.5 from −6.69±2.11 mV to −7.19±2.53 mV upon SHP1i loading.

### Example 2

### SHP1i Release Kinetics Support the use of SWNTs as a Drug Delivery Platform

To mimic in vivo biological conditions and simulate in vivo release, the release profile of SHP1i from SWNT-Cy5.5 in serum was examined (FIG. 1,c). Similar to the release profile in PBS, (FIG. 6), SWNT-Cy5.5 demonstrated sustained release of SHP1 i in serum through 7 days. The drug release profile is nearly linear until day two, with diminishing rates through day seven. The ability of this system to gradually offload substantial amounts of drug over a week suggest it may be suitable for delivering a sustained payload of pro-efferocytic therapy in vivo.

### Example 3

### SWNTs are Taken Up by Macrophages and Enhance Apoptotic Cell Phagocytosis In Vitro

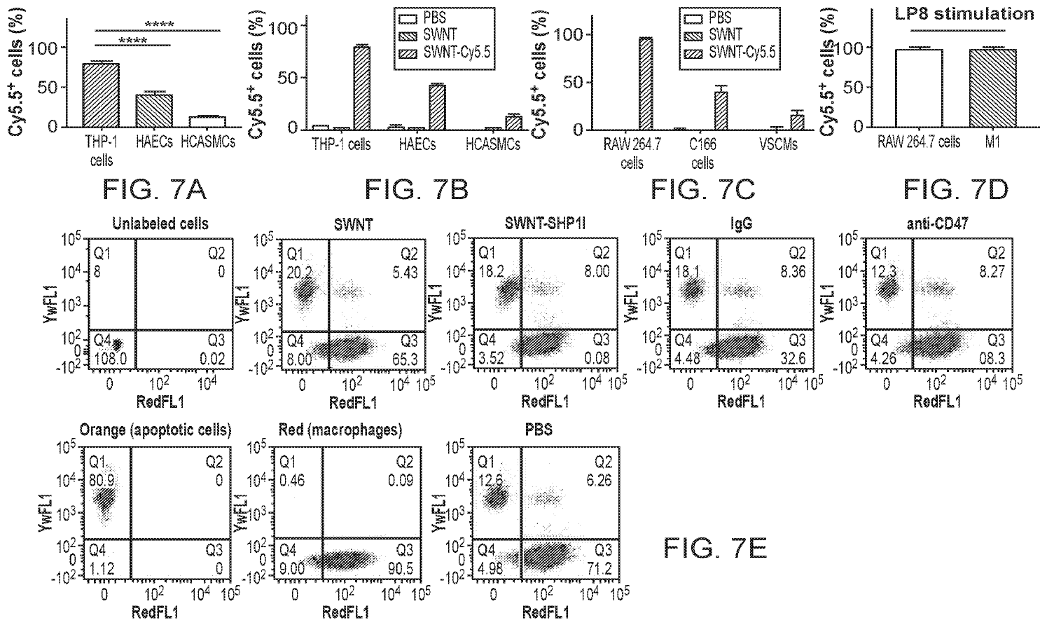

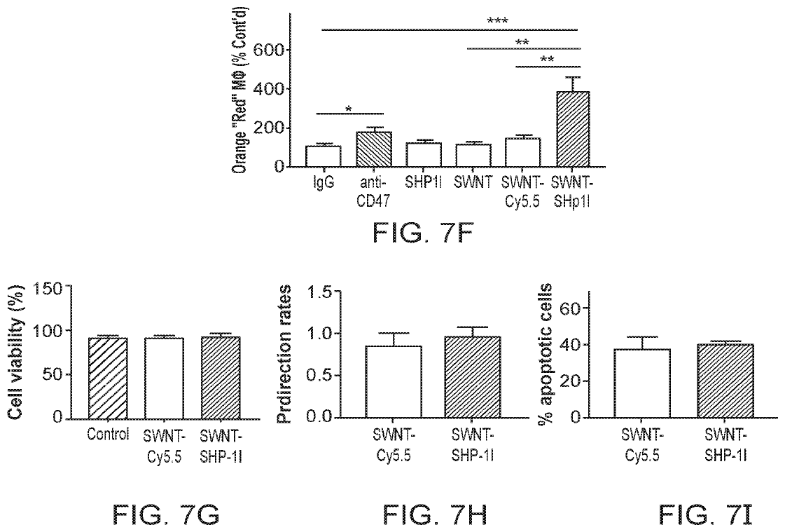

While the exquisite selectivity of SWNTs for circulating Ly-6Chi monocytes was demonstrated, their specificity towards macrophages and other vascular cells was not yet determined. Therefore, the targeting ability of SWNTs was first examined in murine (RAW264.7) and human (THP-1) macrophages and compared to other vascular cells, such as endothelial and vascular smooth muscle cells (VSMCs). Using Cy5.5-positivity as a surrogate for uptake, flow cytometry revealed that SWNTs were robustly and preferentially taken up by >95% of macrophages (FIG. 1,d-e and FIG. 7). Similar targeting efficiency was observed for M1-polarized macrophages, which were stimulated with LPS (FIG. 7).

To confirm their ability to inhibit CD47-induced signaling, the physiologic properties of SWNTs loaded with SHP1i were assessed. In vitro phagocytosis assays confirmed that SHP1i-conjugated SWNTs potently stimulated the clearance of diseased vascular cells exposed to the pro-atherosclerotic factor TNF-a (FIG. 1,f and FIG. 7). Interestingly, compared to anti-1 CD47 antibodies, SWNT-SHP1i yielded the highest degree of apoptotic-cell clearance. Further, SWNT-SHP1i did not alter the cell viability, proliferation rates, or apoptosis of macrophages (FIG. 7). Together, these data indicate that SWNTs stably facilitate the delivery of pro-efferocytic SHP1i specifically to macrophages, enhance their ability to clear apoptotic cells, and do so without altering cell physiology in other ways.

### Example 4

### SWNTs Home to Atherosclerotic Lesions In Vivo

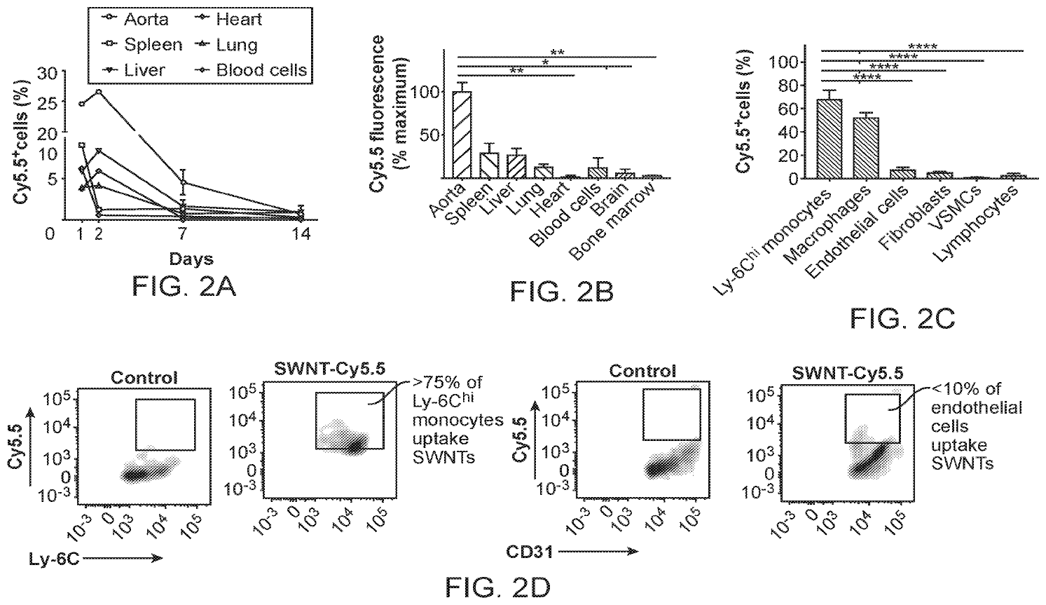

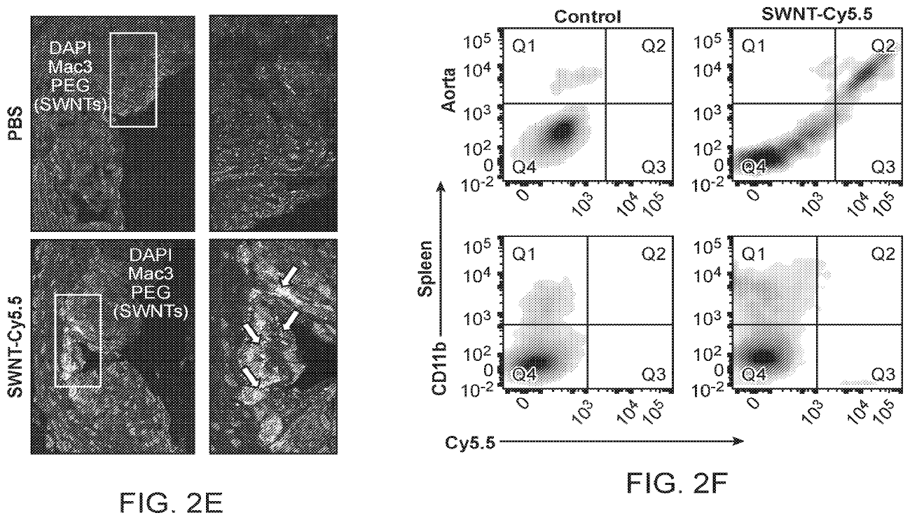

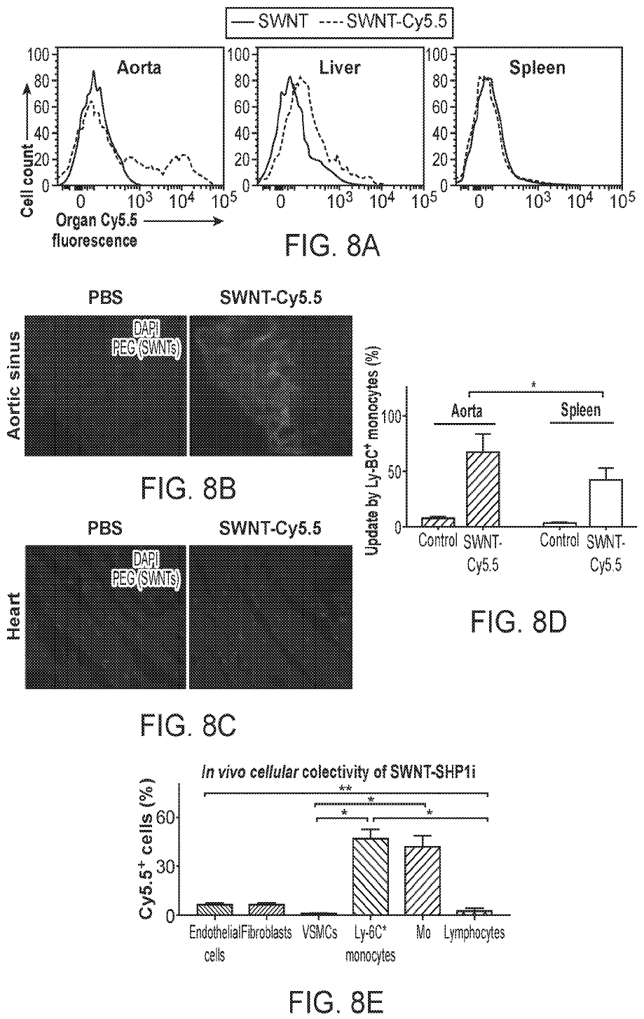

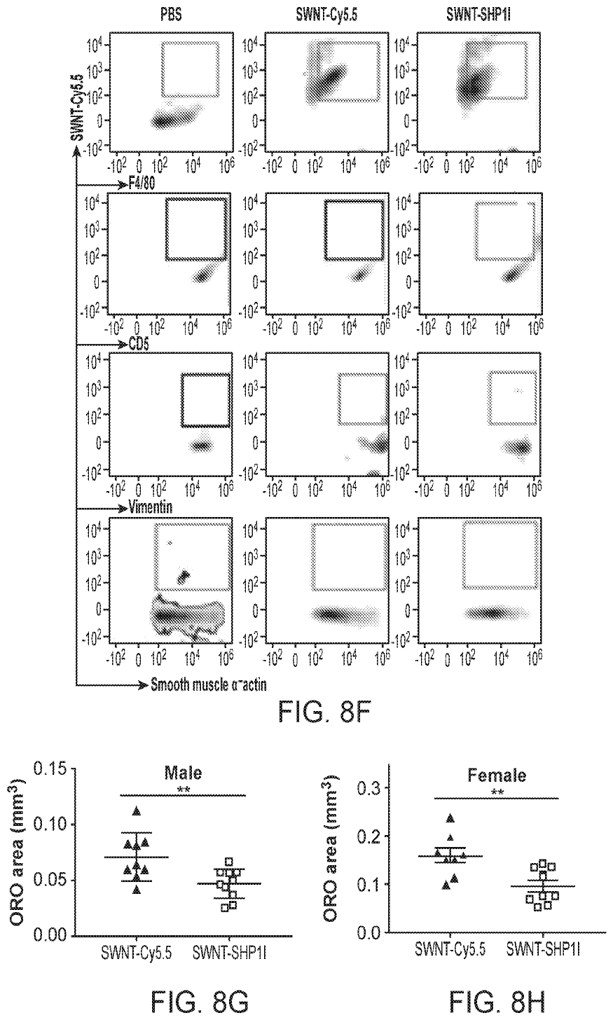

Because a therapeutic agent that is not only taken up by phagocytes, but also delivers the pro-efferocytic payload specifically to the atherosclerotic lesion is highly desireable, the plaque-targeting and biodistribution properties of SWNT nanoparticles were next assessed. apoE^{-/-} mice with established atherosclerosis were systemically administered a single infusion of SWNT-Cy5.5At 24 hr, 48 hr, and 7 days post-injection, flow cytometry analyses of homogenized organs showed that SWNTs preferentially distribute to the atherosclerotic aorta (FIG. 2,a and FIG. 8). Confocal microscopy revealed that SWNTs home to the region with the greatest plaque development, the aortic sinus (FIG. 8). Consistent with prior reports demonstrating SWNT distribution to organs of the reticuloendothelial system, high SWNT uptake in the liver and spleen was also observed at 24 hr post-injection. Of note, SWNT accumulation in the spleen and liver subsequently decreased and remained less than that of the aortae through 7 days (FIG. 2,b).There was also minimal SWNT accumulation in the bone marrow, blood, and vital organs (FIG. 2,b), indicating a favorable biodistribution.

### Example 5

### SWNTs are Preferentially Taken Up by Lesional Macrophages

To identify the specific vascular cell type in which SWNTs accumulate in vivo, high-dimensional 9-color flow cytometry of digested atherosclerotic aortae was performed. The selectivity of SWNTs for inflammatory Ly-6C^{hi} monocytes has been reported in multiple non-vascular mouse models where >99% of circulating monocytes were shown to rapidly internalize SWNTs 2 hr following administration, while <3% of other immune cells took up SWNTs. In an atherosclerosis model, flow cytometry revealed that SWNTs chronically accumulate in Ly-6C^{hi} monocytes and macrophages. After four weeks of weekly SWNT administration, ˜70% of Ly-6C^{hi} monocytes and ˜60% of macrophages had taken up SWNTs, whereas <10% of endothelial cells, <5% of fibroblasts, and negligible amounts of VSMCs and lymphocytes showed SWNT uptake (FIG. 2,c-dand FIG. 8). Immunofluorescence microscopy confirmed that SWNTs co-localize with lesional macrophages (FIG. 2,e). As expected, less SWNT uptake was detected in splenic monocytes and macrophages (FIG. 2,f and FIG. 8). There are two major mechanisms that likely explain this pattern of uptake and robust plaque accumulation. First, SWNTs are taken up by circulating monocytes and traffic to the site of vascular inflammation, as occurs during atherogenesis. Second, SWNTs passively target lesional macrophages (e.g. extravasation through disrupted plaque vessels). Altogether, these data indicate that SWNTs achieve targeted accumulation in the desired plaque-resident phagocytes.

### Example 6

### Pro-Efferocytic SWNTs Prevent Atherosclerosis

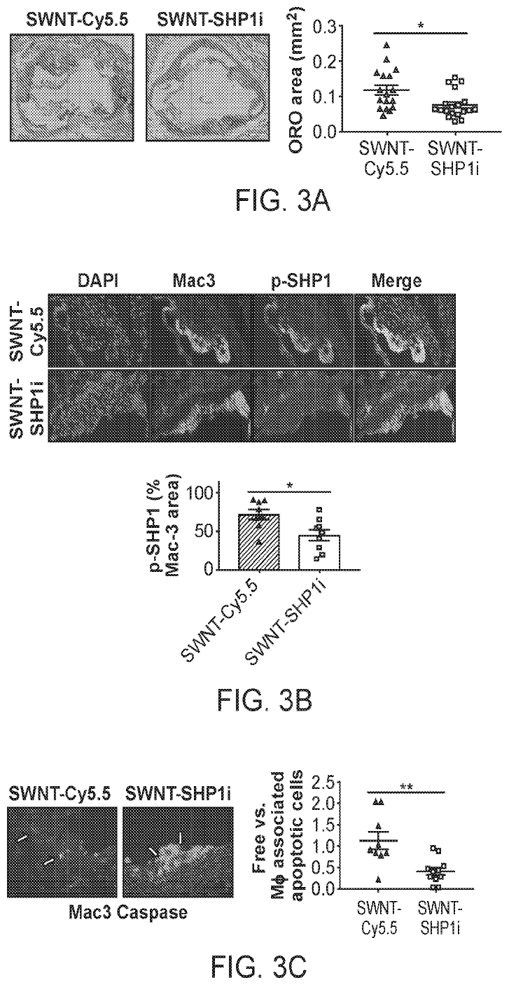

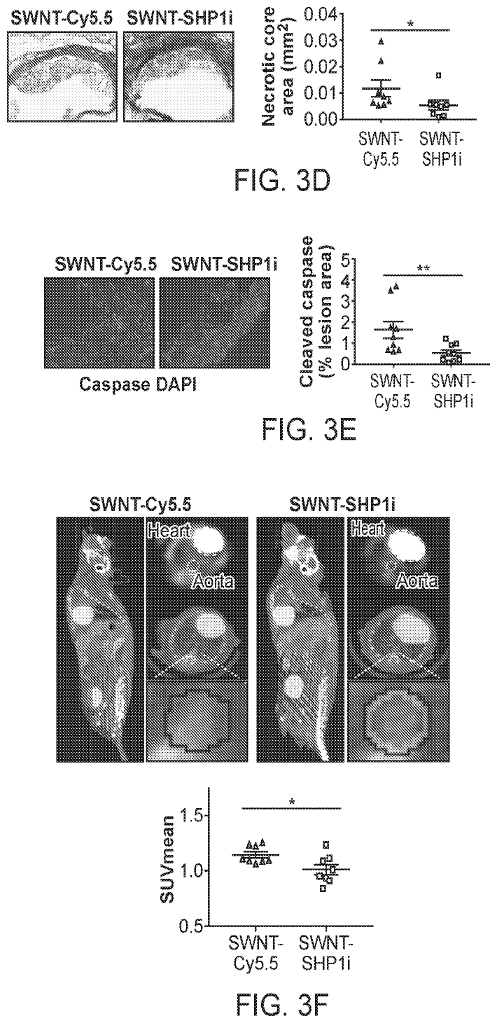

Dyslipidemic apoE^{31 /-} mice subcutaneously implanted with angiotensin II-infusing minipumps to induce atherogenesis were used to assess the therapeutic effect of SWNT-SHP1i on atherosclerosis. Compared to control treatment (SWNT-Cy5.5), treatment with SWNT-SHP1i via weekly injections resulted in a significant anti-atherosclerotic effect (FIG. 3,a and FIG. 8). Analysis of intraplaque SHP-1 activity confirmed that SWNT-SHP1i suppresses the phosphorylation of SHP1i, interrupting the key effector of anti-phagocytic signaling downstream of CD47-SIRPα (FIG. 3,b). To explore efferocytosis in vivo, lesions were assessed for their phagocytic index, or the number of apoptotic cells that were either “free” or associated with macrophages due to efferocytosis. Consistent with findings from in vitro phagocytosis assays, the ratio of free vs. macrophage-associated apoptotic cells was lower in lesions from SWNT-SHP1i animals, indicating enhanced efferocytic activity in the vascular bed (FIG. 3,c). As expected, lesions from SWNT-SHP1i treated mice also displayed smaller necrotic cores (FIG. 3,d) and reduced accumulation of apoptotic bodies (FIG. 3,e).

Efficient efferocytosis also acts to resolve inflammation and prevent the secondary necrosis of dead cells. To assess whether SWNT-SHP1i prevented the inflammatory consequences of defective efferocytosis, in vivo ¹⁸F-fluorodeoxyglucose positron emission tomography/computed tomography (¹⁸F-FDG PET/CT) imaging was performed. Mice treated with SWNT-SHP1i displayed reduced aortic uptake of ¹⁸F-FDG after treatment compared to controls (FIG. 3,f), indicating decreased arterial inflammation. Because persistent inflammation is known to promote plaque vulnerability and the risk for acute cardiovascular events, the apparent ability of pro-efferocytic SWNTs to combat inflammation is particularly intriguing.

### Example 7

### Single-Cell RNA-Sequencing Reveals an Anti-Inflammatory Signature of Macrophages Exposed to Pro-Efferocytic SWNTs

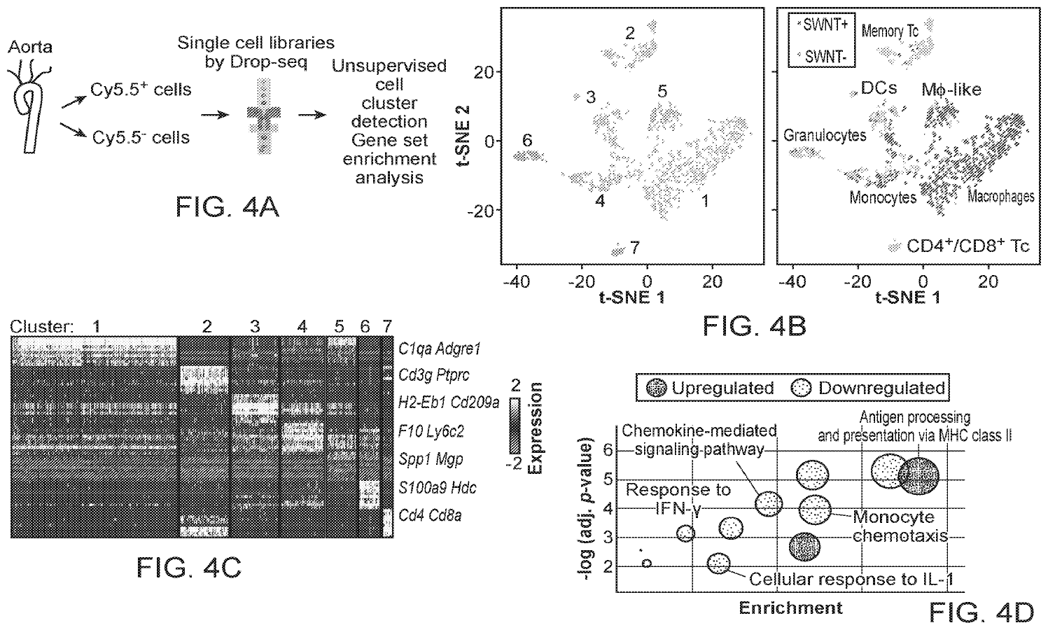

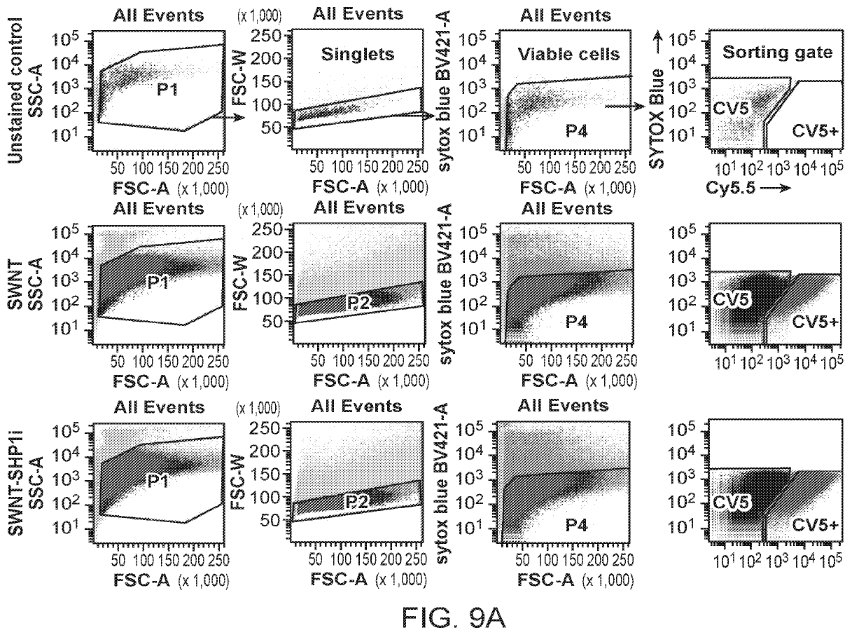

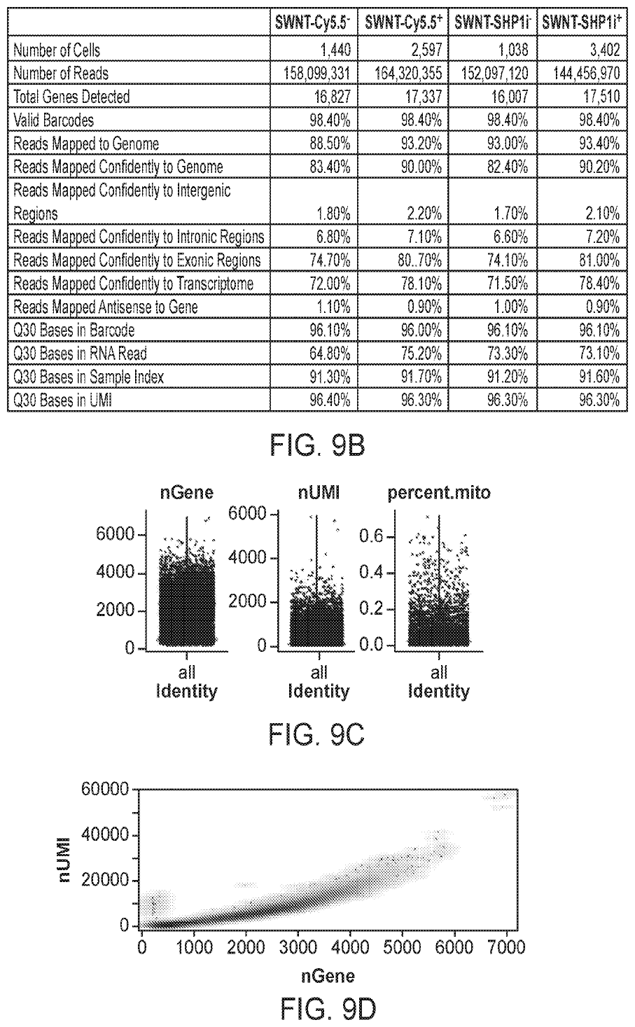

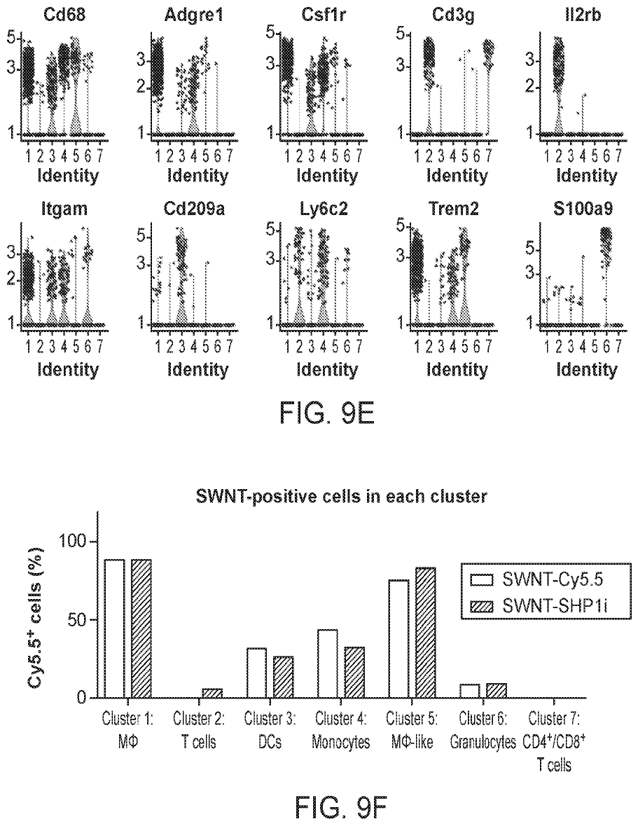

To assess the impact of chronic efferocytosis stimulation on lesional macrophages, large-scale single-cell RNA sequencing (scRNA-seq) was performed on leukocytes from the aortae of SWNT-Cy5.5 and SWNT-SHP1i treated mice. Following fluorescence-activated cell sorting (FACS) for Cy5.5⁺ vs. Cy5.5⁻ cells, single-cell transcriptional profiles were obtained using droplet-based sequencing (FIG. 4,aand FIG. 9). After quality control and filtering, ˜1,500 immune cells were analyzed with a mean of ˜90,000 sequencing reads per cell and expression quantified across 15,309 genes (FIG. 9). Unsupervised clustering grouped cells according to their expression pattern and detected 7 distinct leukocyte clusters in the combined datasets from the aortae of SWNT-Cy5.5 and SWNT-SHP1i treated mice (FIG. 4,b-c). The major cell types were defined according to established immune cell markers and cluster-specific marker genes (FIG. 9), which identified macrophages (Cluster 1), memory T cells (Cluster 2), dendritic cells (DCs, Cluster 3), monocytes (Cluster 4), granulocytes (Cluster 6), and a mix of CD4⁺/CD8⁺ cells (Cluster 7). Cluster 5 contained a “macrophage-like” cell type that expressed myeloid-macrophage markers (Cd68, Lgals3 encoding Mac-2, Trem2 encoding triggering receptor expressed on myeloid cells-2) and genes associated with SMCs and adventitial cells (Spp1 encoding osteopontin, Acta2, Mgp).

Following validation of SWNT selectivity for macrophages by assessing the frequency of Cy5.5⁺ cells in each cluster (FIG. 4,b and FIG. 9), differential expression (DE) analysis was performed to investigate the SWNT-SHP1i-dependent transcriptional response. Gene expression changes in FACS-sorted SWNT-positive cells were compared between treatment groups. SWNT-SHP1i was found to elicit numerous changes in lesional macrophages, including a decrease in pro-inflammatory transcripts (Ccl2, Ccl7, Ccl8, Pf4), and upregulation of genes linked to inflammation resolution (Socs3, Zfp36). Using the identified DE genes, a bioinformatics approach was then applied to explore the upstream regulators and functional significance of such alterations. As expected, both SIRPA (p=3.26×10⁻³) and the SHP-1 encoding gene, PTPN6 (p=4.05×10⁻²) were predicted to mediate the transcriptional changes that occurred. Lesional SWNT-SHP1i treated macrophages were enriched for genes associated with phagocytosis (p=1.78×10⁻⁷) and antigen presentation (p=1.63×10⁻⁷), a process known to be upregulated in macrophages engaged in the clearance of necrotic cells.

Pathway analyses also revealed that SWNT-SHP1i induced an expression signature in macrophages that reflects a decreased inflammatory response (p=5.5×10⁻¹³) and reduced chemotaxis of mononuclear leukocytes (p=2.6×10⁻⁶). Interestingly, Gene Ontology (GO) enrichment analysis further showed that macrophages downregulated genes implicated in the response to interleukin-1 (IL-1, p=8.1×10⁻³) and interferon-gamma (IFN-g, p=7.85×10⁻⁴) (FIG. 4,d). In accordance with previous observations from PET/CT imaging, it appears that targeted efferocytosis stimulation may reduce vascular inflammation without resulting in serious complications like immunosuppression, as described below.

### Example 8

### Pro-Efferocytic SWNTs have a Favorable Safety Profile In Vivo

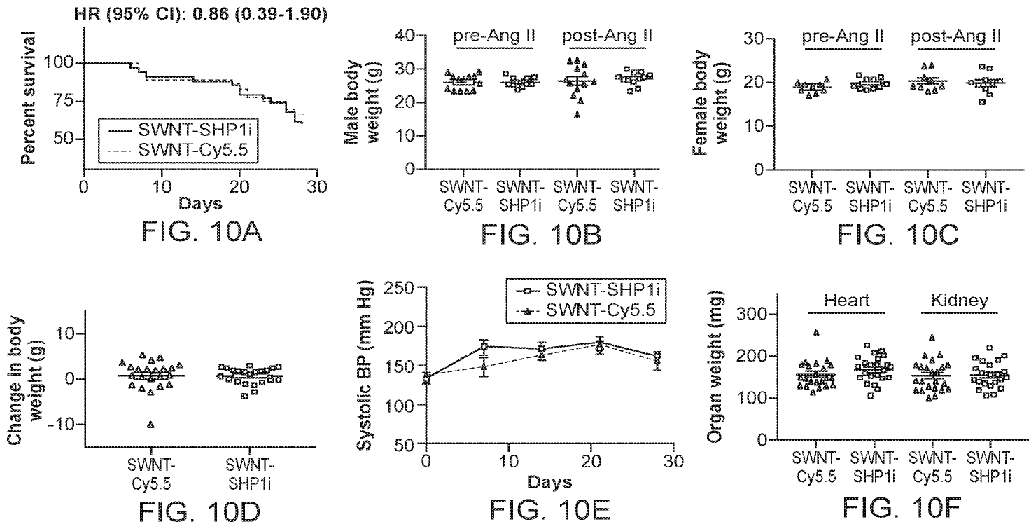

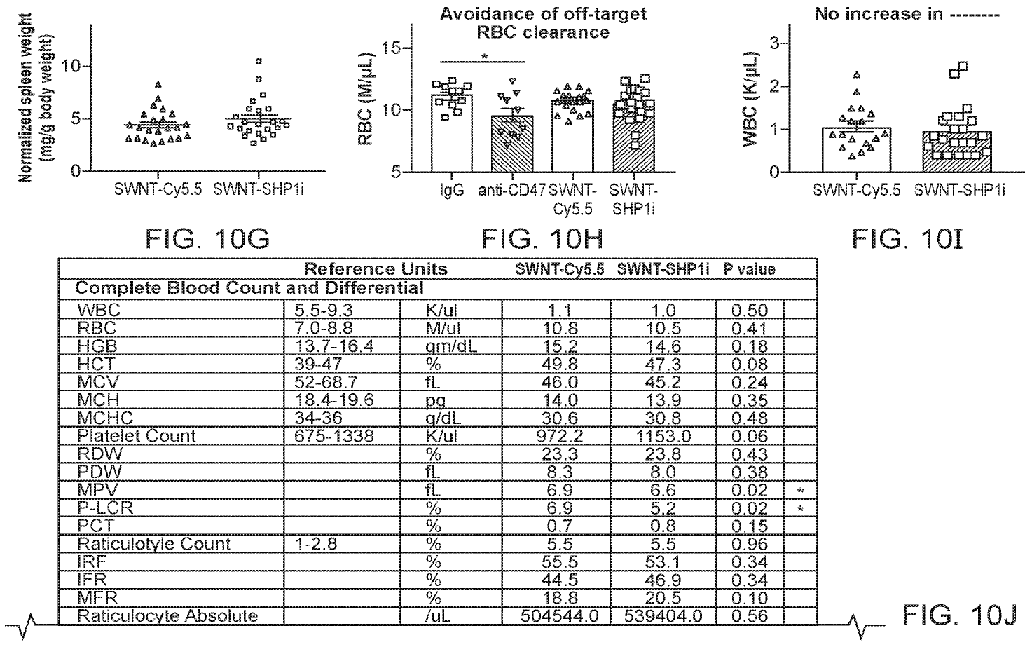

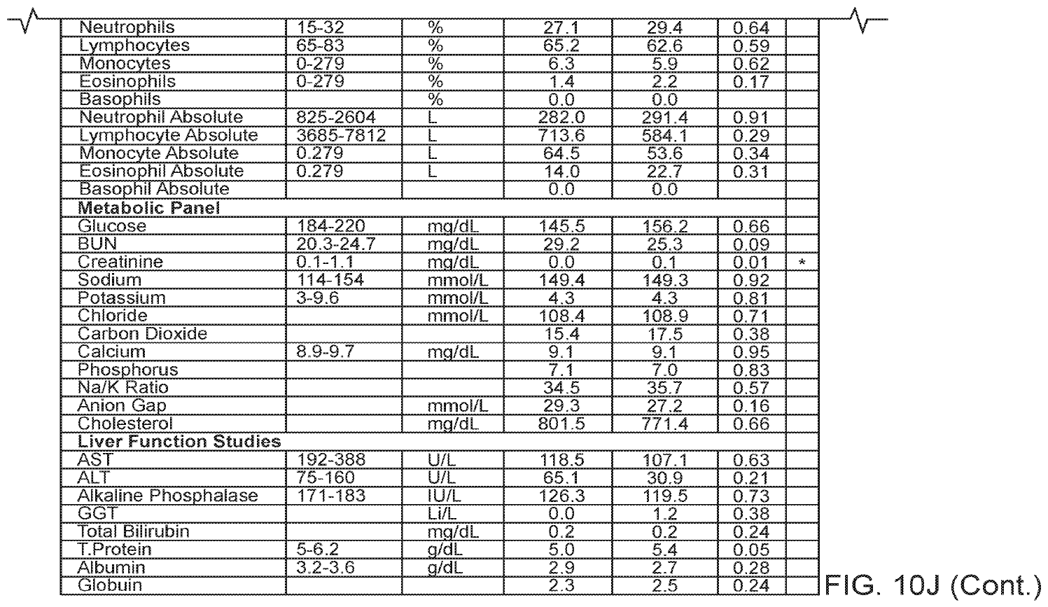

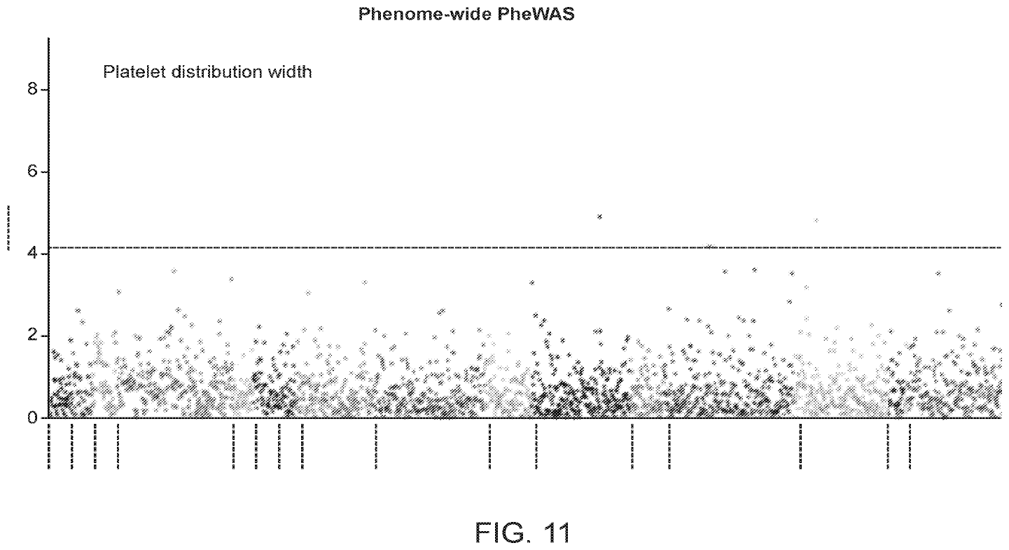

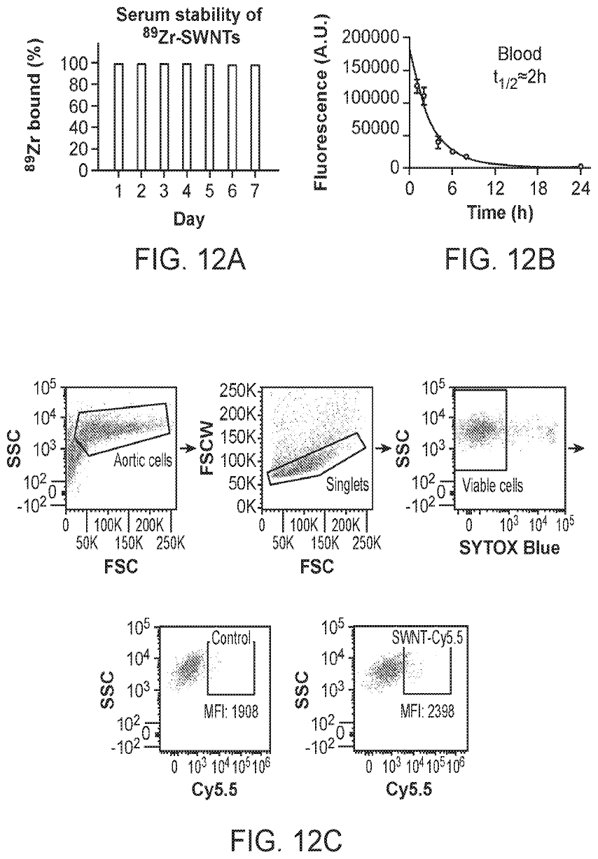

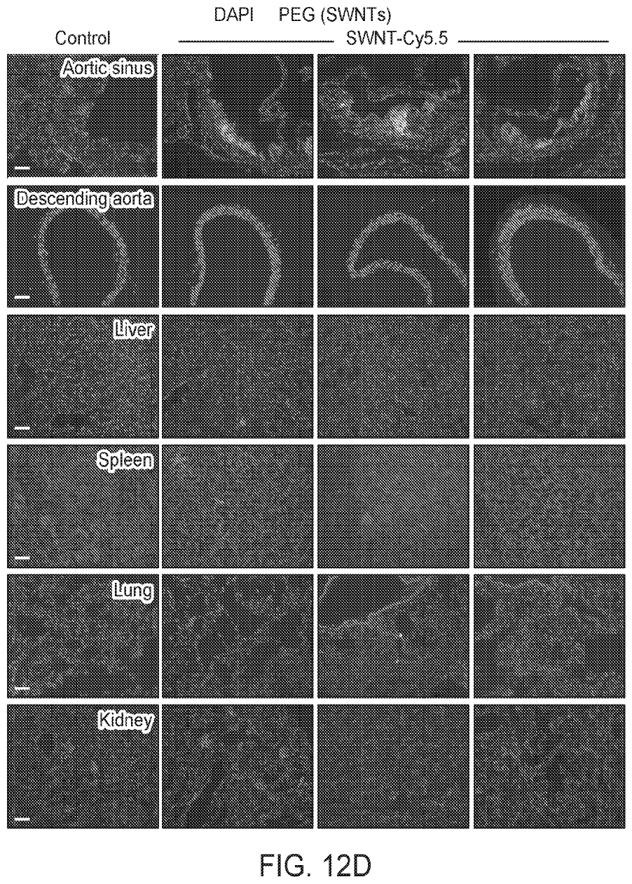

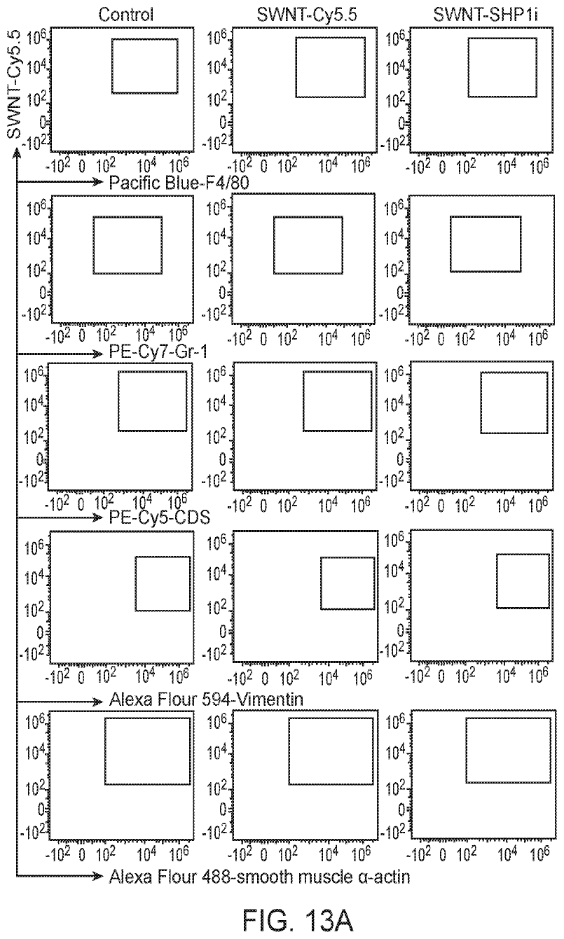

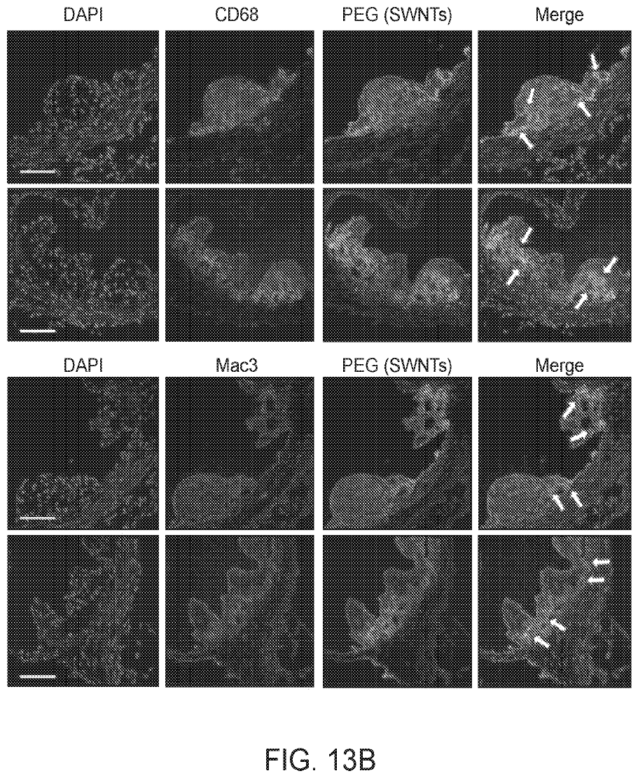

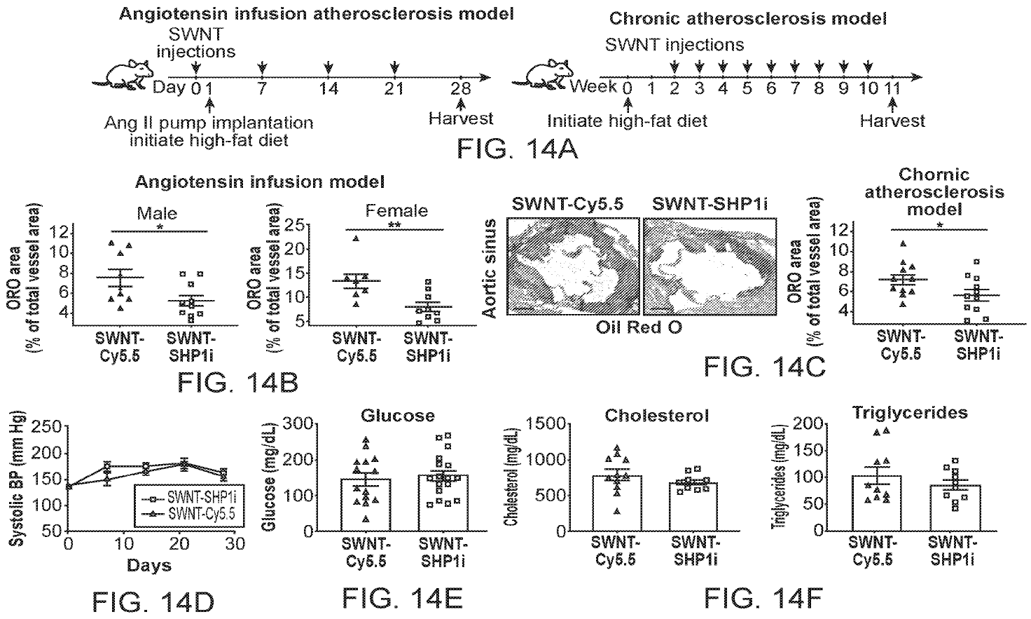

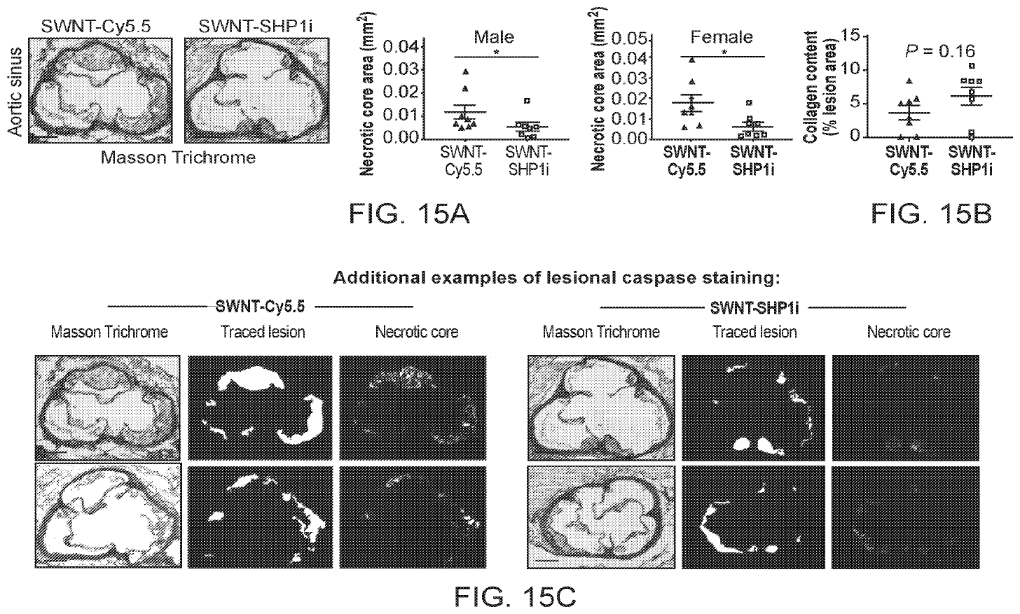

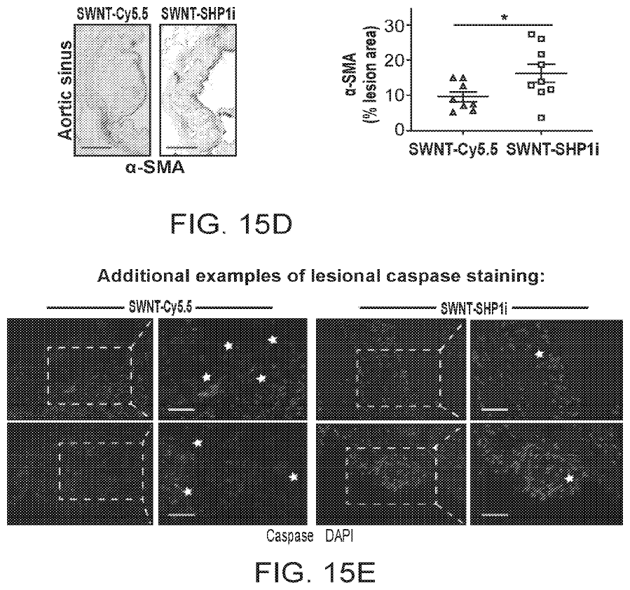

Given that pro-efferocytic antibodies are compromised by adverse effects such as anemia, the safety profile of SWNT-SHP1i was formally assessed. Previous studies have shown that similarly PEG-functionalized SWNTs do not cause acute or chronic toxicities in mice, encouraging further exploration of their applications in medicine. SHP1i-conjugated SWNTs also appeared to be biocompatible and well-tolerated (FIG. 10). Clinical hematology and chemistry results from SWNT-SHP1i treated mice demonstrated no alterations in blood count or metabolic parameters, although there was a decrease in the platelet indices platelet:large cell ratio and mean platelet volume (FIG. 10). These indices are generally interpreted clinically in the context of thrombocytopenia or thrombocytosis. Platelet levels of SWNT-SHP1i treated animals, however, were in the normal range and there was no difference in bleeding or clotting events observed between treatment groups. SWNT-SHP1 i treatment was also not associated with an increase in leukopenia, neutropenia, or clinical infections, and procalcitonin, a biomarker used to identify bacterial infection and sepsis, was unchanged in SWNT-SHP1i treated animals (SWNT-Cy5.5: 0.69 vs. SWNT-SHP1i: 0.76; p=0.2).

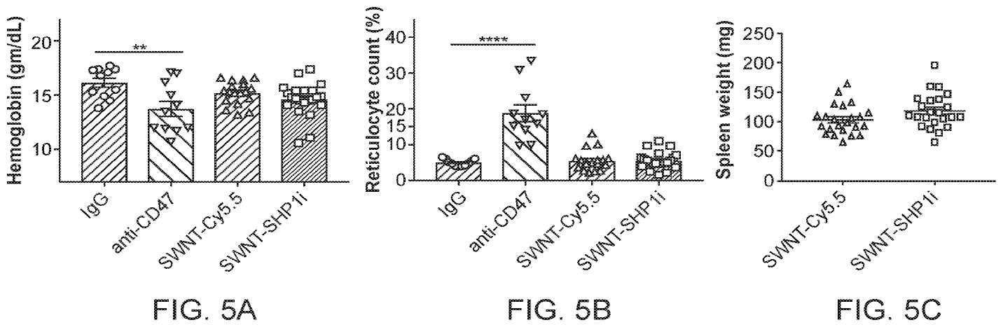

Notably, SWNT-SHP1i treatment was not associated with anemia, the major complication impeding the translation of pro-efferocytic antibodies (FIG. 5,a). Mice did not develop a compensatory reticulocytosis or splenomegaly (FIG. 5, b-c and FIG. 10), as occurs in response to indiscriminate (systemic) blockade of CD47 signaling due to splenic erythrophagocytosis. Of note, SHP-1 is primarily expressed in hematopoietic cells, where it negatively regulates multiple pathways in the immune response. Global SHP-1 deficiency is known to cause defects in hematopoiesis. Given that SWNT-SHP1i treatment did not cause abnormalities in the complete blood count parameters, these data are consistent with the ability of SWNTs to avoid off-target effects and achieve targeted delivery of SHP1i to monocytes and macrophages.

### Discussion:

Cardiovascular disease remains the world's leading killer. Most currently-available therapies only target traditional risk factors (such as hypertension and hyperlipidemia) and do not specifically inhibit the intrinsic, disease-causing pathways known to be active in the vessel wall. Because the “inflammatory hypothesis” of atherosclerosis has now been definitively established, and because robust genetic causation studies have implicated defective efferocytosis as a key driver of plaque expansion, new orthogonal therapies for these risk factor-independent pathways are sought. While major progress has been made in developing agents which can suppress lesional inflammation (e.g. anti-IL-1β antibodies) and/or reactivate engulfment of apoptotic debris in the necrotic core (e.g. anti-CD47 antibodies), each of these approaches has an Achilles heel which may limit its translational relevance. For example, the CANTOS trial revealed that systemic inhibition of the IL-1b pathway potently reduced inflammation and recurrent major cardiovascular events (without altering lipid levels), but unfortunately these benefits were offset by a concomitant increase in fatal infections. Accordingly, more precise targeting of these processes is required if such cutting-edge therapies are to be broadly translated to the cardiovascular realm.

The advent of modifiable, macrophage-specific NPs therefore represents a significant advance in the fight against atherosclerosis. While NPs have been developed for imaging and treatment of atherosclerosis, the lack of sufficient selectivity of the NP to the target cell (e.g. inflammatory monocytes) and desired end organ has hampered their efficacy and utility. By combining innovations in vascular biology and nanotechnology, a “Trojan horse” system was engineered herein, which specifically accumulates in the lesional phagocyte, reactivates efferocytosis locally, and reduces plaque burden, without inducing significant off-target toxicity. Moreover, scRNA-seq data indicate that this approach has the unexpected benefit of suppressing cytokine-dependent vascular inflammation (without the undesirable immunosuppression associated with systemic anti-IL-1β therapy). Recent studies demonstrate the remarkable selectivity of SWNTs for monocytes and macrophages. Understanding of the nanoparticle-cellular interactions that occur and mechanism of SWNT selectivity enables more efficient cell-targeting and delivery to the diseased site. Because the SWNT backbone can be modified to deliver multiple therapeutic agents into the same cell, bi-specific nanoimmunotherapies that simultaneously target efferocytosis and other aspects of macrophage biology (e.g. cholesterol efflux, macrophage skewing) may have a synergistic effect. This platform can also be adapted as a precision therapeutic for the field of immuno-oncology.

### Methods:

SWNT-SHP1i preparation. The functionalized SWNTs were prepared as reported by Smith, B. R. et al. Nat Nanotechnol 9, 481-487, doi:10.1038/nnano.2014.62 (2014), with modifications as follows. Raw HiPco SWNTs (Lot#R0513, Unidym; diameter 0.8-1.2 nm) were added in an aqueous solution of DSPE-PEG₅₀₀₀-amine (NOF Corp) and sonicated for 6 hr, then centrifuged at 100,000g for 1 hr to obtain PEGylated SWNTs. Unbound surfactant was washed by repeated filtration through 100 kDa filters (Millipore). For conjugation of Cy5.5 Mono NHS Ester (GE Healthcare) to SWNT-PEG, Cy5.5 Mono NHS Ester was incubated with SWNT-PEG solution (10:1 mole ratio) for 2 hr. Excess dye was removed by centrifugal filtration. SWNT concentrations were determined spectrophotometrically with an extinction coefficient of 7.9×10⁶ M⁻¹ cm⁻¹ at 808 nm, as reported. High-resolution transmission electron microscopy images were obtained using a FEI Titan 80-300, Environmental TEM (FIG. 6). For SHP1i loading, the SHP1i solution was added to stirred SWNT-PEG-Cy5.5 at 4° C. at pH=7.4 overnight. After 24 hr of stirring, NPs were dialyzed for another 24 hr next to PBS to remove unbound SHP1i molecules. The concentration of loaded SHP1i were measured using nanodrop (Thermo Scietific, Nanodrop2000) at its absorption of 320 nm.

SWNT-SHP1i characterization. To verify the synthesis of SWNT-PEG-Cy5.5-SHP1i, after each step of synthesis, UV-vis spectroscopy and ATR infrared spectroscopy in the 4,000-500 cm⁻¹ region (Nicolet iS50 FT/IR Spectrometer) was performed for PEGylated-SWNTs, SWNT-PEG-Cy5.5, SWNT-PEG-Cy5.5-SHP1i, and SHP1i. Data were collected at room temperature. The surface charge of SWNT-PEG-Cy5.5 and SWNT-PEG-Cy5.5-SHP1i were recorded in deionized water using a ZetaSizer Nano ZS (Malvern Instruments, Worcestershire, U.K.)

Study of SHP1i release from SWNTs. Solutions of SWNT-PEG-Cy5.5-SHP1i were dialyzed at room temperature with PBS (pH=7.4) The spectroscopic absorption of the outlet buffer was measured at different timepoints to quantify the release profile of SHP1i. For study of physiologically-relevant release, solutions of SWNT-PEG-Cy5.5-SHP1i were dialyzed at room temperature with 20% serum solution in PBS. The amount of released SHP1i was measured using UV spectroscopy by sampling the serum solution at different timepoints.

Cell culture. Mouse macrophages (RAW264.7, ATCC TIB-71) and mouse yolk sac endothelial cells (C166, ATCC CRL-2581) were grown in DMEM with 10% fetal bovine serum (FBS), while human monocyte cell line (THP-1, ATCC TIB-202) were grown in RPMI-1640 medium containing 10% FBS and 0.05 mM 2-mercaptoethanol. To induce differentiation of THP-1 monocytes to macrophages, cells were treated with PMA (50 ng/ml) for 24 hrs. Primary vascular SMCs were harvested from the aortae of C57BI/6 mice as described and propagated in DMEM with 10% FBS. Human coronary artery SMCs (HCASMCs, Lonza CC-2583) and human aortic endothelial cells (HAECs, Lonza CC-2535) were cultured and maintained according to the manufacturer's (Lonza) instructions. All cells were cultured in a humidified 5% CO2 incubator at 37° C.

SWNT in vitro uptake assay. Cells were plated in 24-well plates (Corning) until approximately 70% confluent and then incubated with SWNT-Cy5.5 (4nM) for 3 hr in serum-free media at 37° C. Plain SWNTs and PBS-treated cells served as negative controls. After washing cells with PBS, cells were collected and analyzed by flow cytometry (Scanford cell analyzer, Stanford Shared FACS facility). Dead cells were excluded by using SYTOX Blue dead cell stain (Molecular Probes). The rate of SWNT uptake was evaluated by quantifying the percentage of Cy5.5+ cells using FlowJo10.1.r5 (Tree Star, Inc.).

Efferocytosis assay. In vitro phagocytosis assays were performed as described. RAW264.7 macrophages were labelled with CellTracker Red (1 μM, Life Technologies) and pre-treated with SWNT (4 nM), SWNT Cy5.5 (4nM), SWNT-SHP1 (4 nM), or SHP1i (300 nM) for 30 minutes in serum-free media. For target cells, RAW246.7 cells or primary vascular SMCs were labelled with CellTracker Orange (1.2 μM, Life Technologies) and incubated with TNF-α (50 ngmL⁻¹, R&D) for 24 hr to induce apoptosis. Apoptotic cells were then harvested, manually counted, and plated in 24-well dishes at a density of 1.5×10⁵ cells per well. RAW264.7 cells were added to cultured apoptotic cells at 3×10⁵ cells per well and allowed to co-incubate for 2 hr in serum-free media. Anti-CD47 antibody (10 μg/mL, MIAP410, BioXcell) was also tested as a positive control. Following three washes with PBS, cells were dissociated from wells and analyzed by flow cytometry (Scanford cell analyzer, Stanford Shared FACS facility). Efferocytic activity was evaluated as the percentage of phagocytes that were double-positive cells using FlowJo10.1.r5 (Tree Star, Inc.).

Apoptosis assay. RAW264.7 cells were plated at 5,000 cells/well in glass bottom culture dishes 1 (MatTek Corporation), grown overnight at 37° C., and serum-starved for the next 24 hr. Apoptosis was induced with staurosporine (STS, 1 μM, Sigma-Aldrich) in the presence or absence of SWNT-Cy5.5 (4 nM) or SWNT-SHP1 (4 nM) for 2 hours. Apoptosis was evaluated using the In Situ Cell Detection Kit, Fluorescein (Roche, 11684795910), according to the manufacturer's protocol.

Proliferation assay. A modified MTT (3-[4,5-dimethyl-thiazol-2-yl]-2,5-diphenyltetrazolium bromide) assays was performed to analyze macrophage proliferation and viability. RAW 264.7 cells were plated at 25,000 cells/well in a 96-well plate grown overnight at 37° C., and serum-starved for the next 24 hr. Cells were stimulated with 1 10% FBS for 24 hr with or without SWNT-Cy5.5 (4nM) or SWNT-SHP1 (4 nM), and then incubated with 10 uL of MTT AB solution (Millipore, Billerica, Mass.) for 2hr. 100 μl of acidic isopropanol (0.04N HCl) was added to each well and the absorbance was measured at 570 nm (reference wavelength 650 nm) on an ELISA plate reader using SpectraMax 190 Microplate Reader (Molecular Devices).

Experimental animals. Apolipoprotein-E-deficient (apoE^{-/-}) mice on a C56BL/6 background were purchased from the Jackson laboratory and used in the following studies. A total of 105 male and female apoE^{-/-} mice were included. All animals were randomly assigned to the experimental groups. For organ biodistribution studies, male apoE^{-/-} mice were initiated on high-fat Western diet (21% anhydrous milk fat, 19% casein, and 0.15% cholesterol, Dyets Inc.) at 20-24 weeks of age and maintained on this for 4 weeks. For atherosclerosis intervention studies, 8-10 week old apoE^{-/-} mice were implanted with subcutaneous osmotic minipumps (Alzet, model 2004) containing Angiotensin II (angII, VWR, 1000 ng/kg/min) and initiated on a high-fat Western for the ensuing 4 weeks, as described. Both male and female animals were included as per recent NIH policy (Consideration of Sex as a biological variable, NOD-15-102). The “angiotensin infusion” atherosclerosis intervention model was also used in the cellular specificity studies, ¹⁸F-FDG PET/CT imaging, and scRNA-seq. Animal studies were approved 1 by the Stanford University Administrative Panel on Laboratory Animal Care (protocol 27279) and conformed to the NIH guidelines for the use of laboratory animals.

Plaque targeting and biodistribution. apoE−/− mice were injected a single dose of SWNT-SHP1i, SWNT-Cy5.5, or plain SWNTs (without Cy5.5) via tail vein, at a dose previously studied (0.068 mg/ml SWNTs, 200 μL). Mice were euthanized after 24 hr, 48 hr, 7 days, and 14 days and the peripheral blood, bone marrow, aortae, and visceral organs were collected and processed for flow cytometry analysis. Red blood cells were removed from the peripheral blood with ammonium-chloride-potassium (ACK) lysis buffer (Life Technologies). The aortae and visceral organs were homogenized and digested with Liberase™ (Roche, 2 U/ml) and Elastase (Worthington, 2 U/ml) in Hank's balanced salt solution (HBSS) at 37° C. for 1 hr. Digested tissue was passed through a 70-μm strainer to obtain single cell suspensions in 1% BSA/PBS. Fluorescence was detected by flow cytometry (Scanford cell analyzer and Becton Dickinson LSRII) and analyzed using FlowJo10.1.r5.

SWNT cellular uptake profile. Single cell suspensions from the aortae and spleen were obtained as described above and incubated with anti-CD16/32 (BD Biosciences, 553142) for 30 minutes and stained on ice for 30 minutes with the following antibodies: Alexa Fluor 594-anti-Vimentin (clone EPR3776, Abcam, ab154207), APC-anti-CD31 (clone 390, Invitrogen, 17-0311-80), FITC-anti-Ly-6C (clone AL-21, BD Biosciences, 553104), PE-Cy5-labeled anti-CD5 (clone 53-7.3, BioLegend, 100609), PE-Cy7-anti-Gr-1(clone RB6-8C5, Invitrogen, 25-5931-81), APC-Cy7-anti-CD11b (clone M1/70, BioLegend, 101225), and Pacific Blue-anti-F4/80 (clone BM8, BioLegend, 123123). For intracellular staining, cells were fixed and permeabilized with buffers (BD Phosflow Fix Buffer I and Perm Buffer III) according to the manufacturer's instructions, then stained with Alexa Fluor 488-anti-alpha-smooth 1 muscle actin (clone 1A4, eBioscience, 50-112-4644). Cell suspensions were subjected to flow cytometry (Becton Dickinson LSR II) and analyzed using FlowJo10.1.r5.

Atherosclerosis intervention studies. To evaluate the therapeutic effect of pro-efferocytic SWNTs, angiotensin-infused apoE^{-/-} mice were treated with either SWNT-Cy5.5 (n=20 male, 13 female) or SWNT-SHP1 (n=21 male, 15 female) i.v. weekly for 4 weeks (0.068 mgml-1 SWNTs, 200 μL). SWNT therapy began one day before osmotic pump implantation. Body weights were evaluated before and after treatment. Animals were observed daily, and in the case of premature sudden death, necropsy was performed to determine the cause of death. Blood pressure was measured in conscious mice at baseline and on a weekly basis throughout the study period using the Visitech BP-2000 System (Visitech Systems). Following 4 weeks of treatment, mice were sacrificed after an overnight fast, with their aortae, peripheral blood, and visceral organs collected. The weights of the spleen, heart, and kidney were recorded, as well as those of any unusually-sized organs. Complete blood count, metabolic panel, and lipid profile determinations were performed by the Animal Diagnostic Laboratory in the Stanford Veterinary Service Center.

Tissue preparation and immunohistochemical analysis. For aortic root analysis, mice were perfused with PBS via cardiac puncture in the left ventricle and then perfusion fixed with phosphate-buffered PFA (4%). Aortic roots and visceral organs were collected, embedded in OCT, and sectioned at 7-μm thickness, starting from the base of the aortic root and covering the entire aortic sinus area. For analysis of atherosclerotic burden, four tissue sections at 100-μm intervals were collected from each mouse and stained with ORO (Sigma Aldrich, O1516). Lesion area was quantified from the luminal aspect of the blood vessel through the plaque to the internal elastic lamina (e.g. lipid in the neointima was quantified). Necrotic core size was quantified by calculating the area of the lesion that was acellular on Masson's trichrome staining (Sigma Aldrich). To 1 assess lesional SHP-1 activity, sections were co-stained with phospho-SHP1 (Abcam, ab131500, 1:50) and Mac-3 (BD Sciences, BD 550292, 1:100), followed by Alexa Flour 488 and 594 (Life technologies, 1:250), respectively. Phospho-SHP1 area was quantified and normalized to Mac-3 area, as described. To assess apoptotic cells (ACs) in lesions, sections were stained for cleaved caspase-3 (Cell Signaling, 9661, 1:200) staining followed by Alexa Fluor 488 goat anti-rabbit (Life technologies, 1:250). The percentage of cleaved caspase-3⁺ area was calculated and divided by the total atherosclerotic plaque area measured by ORO in serial sections. To study the phagocytosis of ACs by macrophages, the in vivo phagocytic index was calculated, as described. Sections were co-stained with cleaved caspase-3 and Mac-3, followed by Alexa Flour. The phagocytic index was determined by manually counting the number of free ACs versus phagocytosed ACs. ACs were considered free if they were not associated with a macrophage. For detection of SWNTs, sections were stained with anti-PEG (Abcam, PEG-B-47, ab51257, 1:100). Additional quantitative analyses were performed with aortic lesion sections stained with smooth muscle α-actin (SMA, Abcam, ab5694, 1:300), Mac-3 (BD Sciences, BD 550292, 1:100), CD-3 (Abcam, ab5690, 1:150), and Ly-6G (BD Sciences, BD 551459, 1:300). SWNT co-localization images were taken on an inverted Zeiss LSM 880 laser scanning confocal microscope. All other images were taken with a Nikon digital camera mounted on a fluorescent microscope and analyzed using Adobe Photoshop CS6 in a blinded fashion.

In vivo PET/CT imaging. ¹⁸F-FDG-PET/CT imaging was used to assess changes in atherosclerotic inflammation in response to treatment with SWNT-SHP1i or control SWNT-Cy5.5 (n=8 per group). The mice were fasted overnight prior to the scan. Special precautions were taken during isoflurane-induced anesthesia to maintain body temperature (before injection, after injection and during the scan). The radiotracer (15-20 MBq of ¹⁸F-FDG; Stanford Cyclotron & Radiochemistry Facility) was administered intravenously to the mice. In addition, a long circulating formulation of iodinated triglyceride (Fenestra VC, MediLumine, Canada) was used as contrast agent. 3 hr after ¹⁸F-FDG administration, the mice 1 were placed on the bed of a dedicated small animal PET-CT scanner (Inveon PET/CT, Siemens Medical Solution, USA), and a 30-min static PET scan was obtained. All images were reconstructed using OSEM. The same acquisition bed was used for the CT scan. The CT system was calibrated to acquire 360 projections (voltage 80 kV; 5 current 500 μA). The voxel size was 0.206×0.206×0.206 mm³. Region-of-interest (ROI) analysis was performed using IRW software (Inveon Research Workplace, Siemens, Germany). ¹⁸F-FDG uptake in the thoracic aorta was quantified by drawing 3D ROls on the axial slices from the CT scan. The standardized uptake values (SUVs) were calculated and the mean value was used.

Aortic single cell preparation for single-cell RNA sequencing. Aortae (including the aortic sinus and aortic arch) were carefully dissected free from the perivascular adipose tissue and cardiac muscle, and then digested into single cell suspensions as described above. Cells were pooled from mice treated with SWNT-SHP1i (n=4) and SWNT-Cy5.5 (n=4), and stained with SYTOX Blue (Invitrogen, S34837) to discriminate and exclude non-viable cells. Viable cells (SYTBOX Blue⁻) were sorted with a 100-μm nozzle into populations that were Cy5.5⁺ and Cy5.5⁻ using a BD Aria II and collected in PBS+0.04% BSA.

Single-cell RNA sequencing. Samples were resuspended to a concentration of 600-1000 cellsμ⁻¹ in PBS+0.04% BSA and loaded into the 10× Chromium system to generate single-cell barcoded droplets using the 10× Single Cell 3′ reagent kit v2 (10× Genomics), according to the manufacturer's protocol. Briefly, each single cell is encapsulated in a gel bead that is functionalized with a 14-base pair (bp) cell-specific barcode and 10-bp unique molecular identified (UMI) that tags each mRNA molecule. Reverse transcription occurs in each gel bead. The 10X system utilizes droplet-based microfluidics to capture the mRNA of thousands of cells in parallel. Following capture and lysis, full-length cDNA is synthesized and amplified for construction of Illumina sequencing libraries. The resulting libraries were sequenced across three 1 lanes on an Illumina HiSeq4000 platform with a 26-bp Read 1 to sequence the cell barcode and UMI, an 8-bp i7 index read to sequence the sample index, and a 98-bp Read 2 to sequence the transcript using a paired-end configuration. Library preparation and sequencing was performed by the Stanford Functional Genomics Facility.

scRNA-seq data analysis. Single cell RNA-sequencing data was pre-processed using 10× Cell Ranger software (Cell Ranger v3.0.2), including data de-multiplexing, barcode processing, alignment, and single cell 3′ gene counting, as described. Reads that were confidently mapped to the reference mouse genome (UCSC mm10) were used to generate a gene-barcode matrix for downstream analysis. The filtered gene-barcode matrices containing only cell-associated barcodes were merged into a combined matrix from the above control (SWNT-Cy5.5) and treated (SWNT-SHP1i) datasets. Genes expressed in <5 cells, cells with <200 or >4,000 detected genes, and cells with a percentage of mitochondrial genes >6% were filtered. After additionally filtering adventitial cells, 1,274 immune cells were included to assess the effect of chronic inhibition of the CD47-SIRPa-SHP1 axis. The resulting data was log normalized, scaled, and regressed on the number of UMIs per cell and the percentage of mitochondrial gene content. Principle component analysis (PCA) was performed for dimensionality reduction using the top 1,000 variable genes ranked by their dispersion from the combined datasets, followed by unbiased clustering analysis based on the identified PCs and t-Distributed Stochastic Neighbor Embedding (t-SNE) for data visualization. To identify cell-type specific responses to SWNT-SHP1i treatment, differential expression tests were performed for cell clusters comparing samples from mice treated with SWNT-SHP1i and SWNT-Cy5.5. DE-genes with p<0.05 based on the Wilcoxon rank sum test were considered statistically significant. All downstream analyses were performed with the Seurat R package v3.0.

Pathway analysis. Pathway analysis was performed using significantly up-regulated and down-regulated genes between SWNT-SHP1i and SWNT datasets. Genes were input for pathway analysis by Qiagen Ingenuity Pathway Analysis for upstream regulator analysis and assessment of enriched canonical pathways, diseases, and functions; and PANTHER Pathway for GO term enrichment analysis (Ashburner, et al., 2000) (PANTHER Overrepresentation Test released 2018-11-13, GO Ontology database released 2019-01-01). All p-values were corrected for multiple comparisons (Bonferroni correction). GOPlot 1.0.2 was used for visualization of results from GO enrichment analysis.

Statistical analysis. Categorical data was compared using the Fisher's Exact Test. Continuous data are presented as mean±SEM and were tested for normality using the D'Agostino Pearson test. Groups were compared using the two-tailed Student's t-test for parametric data and the Mann-Whitney U test for non-parametric data. When comparing more than two groups, data were analyzed using ANOVA followed by post-hoc tests. For atherosclerosis intervention studies, survival analysis was performed using the Kaplan-Meier method, with the log rank test used to compare time-to-mortality curves. A p-value <0.05 was considered to indicate statistical significance. Statistical analyses were performed using GraphPad Prism 7 (GraphPad Inc.).

The SWNTs have been synthesized and characterized, and have been loaded with small molecules and antibodies and labeled them with fluorescent dyes. In vitro cell experiments show greater efficacy for this system (i.e., in improving efferocytosis) compared with anti-CD47 antibody treatment and in vivo experiments in an atherosclerosis mouse model. The present compositions and methods disclosed herein successfully reduce atherosclerosis, confirming the utility of this system for atherosclerosis treatment.

### Example 9

Here, we developed and characterized an intracellular phagocytosis-stimulating treatment in the CD47-SIRPα pathway. We loaded a novel monocyte/macrophage-selective nanoparticle carrier system with a small molecule enzymatic inhibitor that is released in a pH-dependent manner to stimulate macrophage efferocytosis of apoptotic cell debris via the CD47-SIRPα signaling pathway. We demonstrated that single-walled carbon nanotubes (SWNTs) can selectively deliver tyrosine phosphatase inhibitor 1 (TPI) intracellularly to macrophages, which potently stimulates efferocytosis, and chemically characterized the nanocarrier. Thus, SWNT-delivered TPI can stimulate macrophage efferocytosis, with the potential to reduce or prevent atherosclerotic disease. This study overcomes previous limitations through the use of a novel monocyte/macrophageselective nanoparticle carrier system (SWNTs) loaded with an intracellular enzymatic inhibitor that releases in a pH-dependent manner to reactivate macrophage efferocytosis.

SWNTs are loaded with tyrosine phosphatase inhibitor 1 (TPI-1, which we term TPI), a potent SHP-1 inhibitor, using non-covalent π-π stacking interactions on SWNTs. TPI is selective toward SHP-1 (IC₅₀ of 40 nM) over SHP-2, another tyrosine phosphatase expressed in cells, and it is more selective than many other SHP-1 inhibitors, such as NSC 87877. SWNT-based immunotherapy provides a safer alternative to anti-CD47 antibodies and associated CD47-blocking strategies while retaining improved rates of efferocytosis due to: i) their selectivity to inflammatory monocytes/macrophages; ii) their capability to enter monocytes/macrophages and deliver small molecule drugs such as TPI intracellularly; and iii) their ability to release loaded drugs in a pH-dependent manner over time. Indeed, drugs are often loaded onto nanoparticles in large payloads for intracellular release of small molecules. While these drugs are commonly covalently linked to their nanoparticle carriers, this can result in drug loading limitations, unexpected pharmacological effects, and release which is difficult to control. Moreover, delivery of nanomaterials to target tissues remains a problem due to heterogeneity in access to the diseased tissue. While many studies have investigated the effects of nanoparticle shape and size on circulation and extravasation rate into disease sites within the body, they have concluded that no single nanoparticle type serves as the best targeting agent for all situations. New approaches based on targeting immune cells for native homing to the plaque may thus reduce heterogeneity in targeting.

SWNTs were developed to avoid these issues with extravasation and heterogeneity, as they are almost exclusively taken up by inflammatory monocytes, which differentiate into the phagocytic macrophages that are capable of clearing cell debris in atherosclerotic plaques. SWNTs' combination with TPI thus makes them potent stimulators of macrophage scavenging in the CD47 pathway. Here, we demonstrate: i) macrophage phagocytosis can be stimulated intracellularly via the CD47-SIRPα signaling pathway by small molecules, unlike other approaches which employ proteins (CD47 antibodies, SIRPα variants); ii) selective SWNTs can deliver small molecules intracellularly to macrophages; ii,) SWNTs release small molecule inhibitors in a pH-dependent manner (thus, release is optimal in the acidic intracellular regions of monocytes); and iv) this delivery approach reactivates macrophage efferocytosis in vitro at least as effectively as anti-CD47 antibodies.

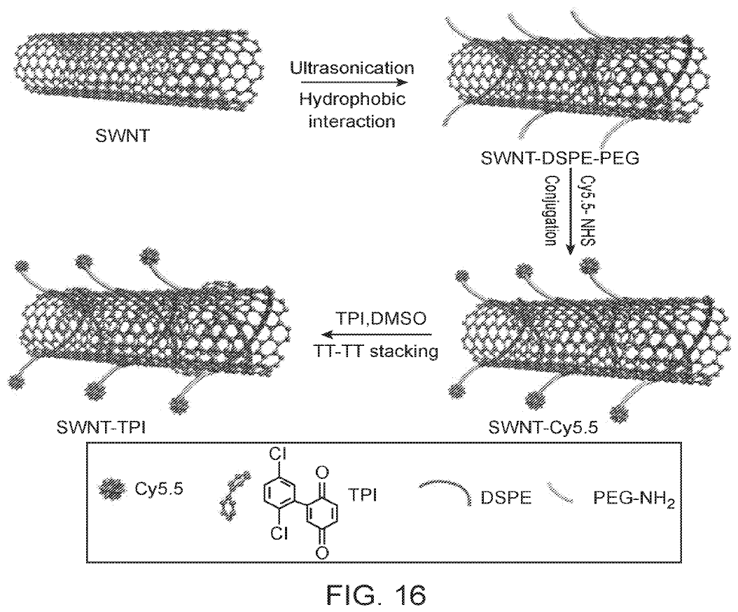

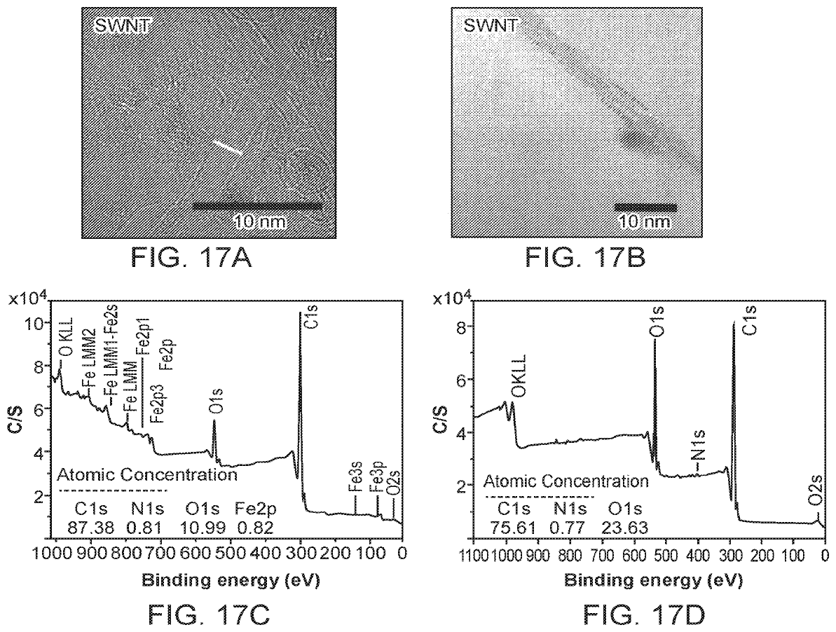

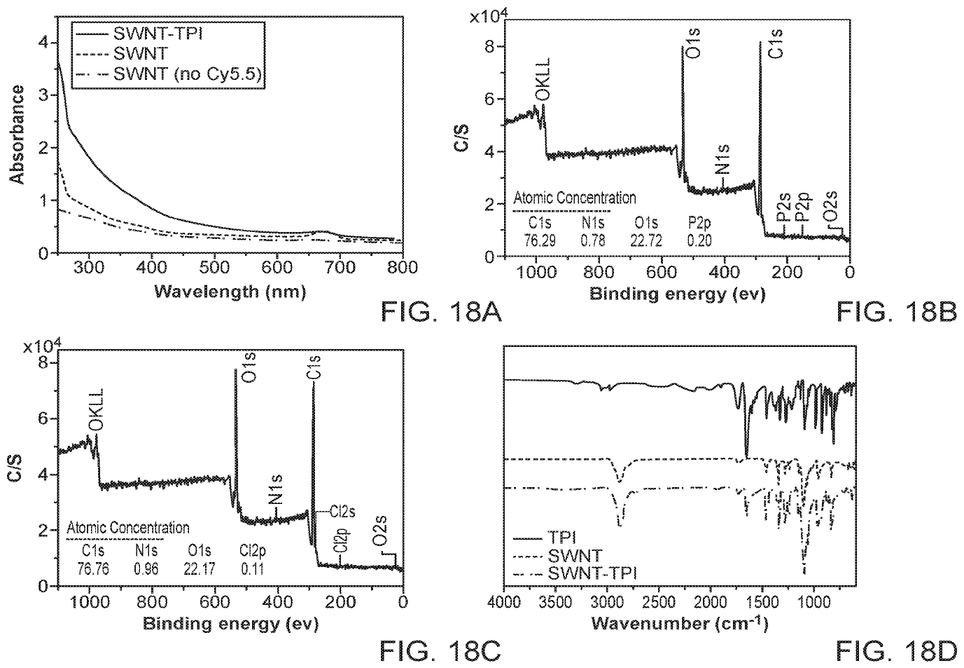

We prepared SWNT nanodelivery vehicles, which comprise a backbone of polyethylene glycol (PEG)-functionalized single-walled carbon nanotubes (SWNTs) conjugated to a fluorescent dye (Cy5.5) and loaded with a small-molecule, TPI, via π-π stacking (FIG. 16). DSPE-PEG adheres to SWNTs via hydrophobic interactions, and we validated the presence of DSPE-PEG on SWNTs (FIG. 16, upper right) using X-Ray Photoelectron Spectroscopy (XPS) (FIG. 17c-2d). The existence of N elements, the C—O groups at 285.9 eV (PEG backbone), and the O—C═O groups at 288.4±0.1 eV, which represent the ester linkages associated with DSPE-PEG molecules, all confirmed the decoration of lipid on SWNTs. We further confirmed the presence of Cy5.5 fluorescent dye (used for flow cytometric characterization) and TPI loaded on SWNTs using a suite of characterization strategies, including UV-Visible spectroscopy, XPS, and Fourier-Transform Infrared spectroscopy (FT-IR) (FIG. 18).

UV-Vis spectroscopy validated the presence of Cy5.5 (sharp peak at ˜674 nm) and TPI (absorption peak at ˜312 nm) (FIG. 18a), and also allowed us to quantify the number of respective molecules per SWNT. The presence of TPI on SWNTs was further validated by XPS and FT-IR. Two CI element peaks at ˜195 and 294 eV can be observed in the SWNTDSPE-PEG-Cy5.5-TPI (which we term SWNT-TPI) sample compared with SWNT-DSPE-PEGCy5.5 (which we term SWNT, SWNT lacking Cy5.5 will be termed SWNT (no Cy5.5)) (FIG. 18b-3c) in XPS charts, revealing the presence of the TPI molecule. In the FT-IR analysis, SWNT-TPI shows two new peaks at ˜3052₋₁ and ˜1655 cm₋₁ compared to SWNTs, validating TPI loading on SWNTs (FIG. 18d).

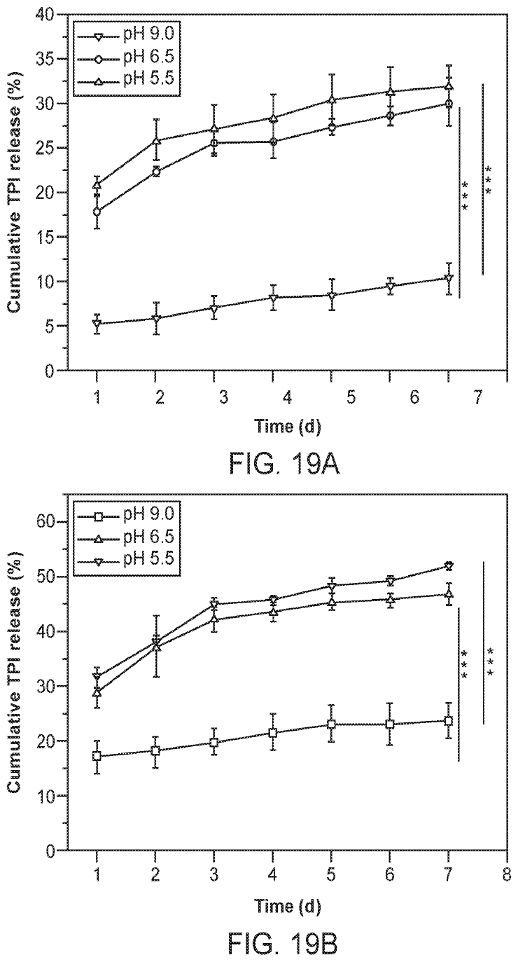

We studied the release profile of TPI from SWNTs in PBS at different pH (6.5 and 5.5), in carbonate buffer (pH 9), and in serum (PBS+20% FBS) across various pH levels. We found that TPI release is accelerated by progressively more acidic conditions, with pH 5.5 and pH 6.5 more rapid than pH 9.0 across time-points through day 7 (FIG. 19a). Thus, TPI release will greatly increase under the acidic conditions of the monocyte/macrophage intracellular milieu where the pH is acidic, ˜5-5.5. Moreover, the slow release at more basic pH suggests our nano-platform features longer-term drug retention on the NPs prior to release.

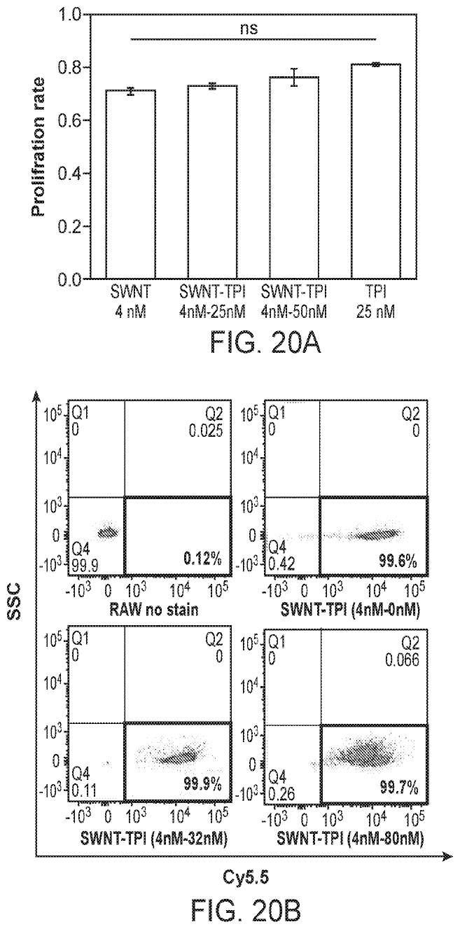

TPI release kinetics in vitro in PBS buffers may be slow due to the relatively low TPI solubility in aqueous buffer. We demonstrated the pH-dependence of TPI release from SWNTs using a serum (20%)-containing buffer. TPI release from SWNTs in serum displays generally similar tendencies to that in PBS; i.e., a more acidic pH results in accelerated release kinetics (FIG. 19b). However, notably, across the range of pH tested, TPI is released more rapidly in serum compared with PBS. Thus, π-π stacking of TPI on SWNTs is advantageous compared to covalent bonding due to decreased toxic metabolites, ease of loading, consistent pharmacological effects, as well as a sensitive, pH-dependent release process. We explored the effect of TPI, SWNTs, and SWNT-TPI on cell health via cell proliferation assays in vitro and observed no significant effect of TPI and SWNT-TPI on RAW cell proliferation compared with SWNTs alone (FIG. 20a). We have previously demonstrated the exquisite selectivity of SWNTs for circulating Ly-6Cₕᵢ monocytes in vivo, and here we confirm the excellent uptake of SWNTs by phagocytic RAW 264.7 cells. After co-culturing SWNTs or SWNT-TPI with RAW cells for 48 hrs, over 99% of RAW cells took up SWNTs (4 nM), independent of TPI concentration (0, 32, and 80 nM) based on flow cytometry analysis (FIG. 20b).

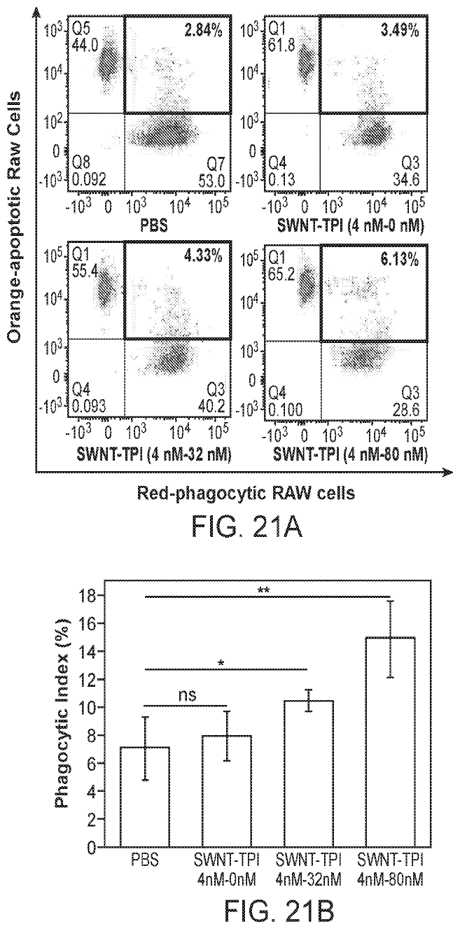

To investigate the ability of SWNT-TPI to stimulate macrophage efferocytosis, we employed in vitro phagocytosis assays. 48 hrs after co-culturing SWNTs or SWNT-TPI with RAW cells, 99% of RAW cells took up SWNTs bearing varied TPI concentrations (0, 32, or 80 nM, holding SWNT concentration constant at 4 nM) (FIG. 21). Then we co-cultured phagocytic RAW cells containing SWNTs or SWNT-TPI with apoptotic RAW cells. We observed significantly more phagocytosis of apoptotic cell debris in the SWNT-TPI (TPI: 80 nM) condition compared with phagocytic RAW cells treated with PBS, SWNTs (4 nM), and SWNT-TPI (TPI: 32 nM) (FIG. 21a), indicating SWNT-TPI enhanced macrophage phagocytic function. Indeed, TPI-concentration dependence was observed, as phagocytic RAW cells in the SWNT-TPI (TPI: 32 nM) condition also significantly, albeit slightly, improved phagocytic function compared with PBS (*P=0.0293), and SWNTs lacking TPI did not. Thus, SWNT-TPI (4 nM-80 nM) displayed the highest phagocytic index (**P=0.005) compared with all groups tested.

Atherosclerotic plaques, driven in part by dysfunctional efferocytosis, are the main cause of cardiovascular disease-related deaths. Stimulating plaque macrophage efferocytosis to remove the debris from necrotic plaque cores is emerging as a promising therapeutic approach. The principles of nanomedicine are adopted given nanomaterials' multifunctional ability to: (1) specifically target certain cell types; (2) carry large amounts of drugs to target; and (3) enable “smart”, environmentally-responsive release properties. SWNTs enter inflammatory monocytes and macrophages with unprecedented selectivity in vivo. Moreover, SWNTs can carry 10 s to 100 s of small molecule drugs per nanoparticle with pH-dependent release.

Here, we take advantage of SWNTs' merits to provide a potentially safer alternative to anti-CD47 antibodies and associated CD47-blocking strategies while retaining improved rates of efferocytosis. Our SWNT-SHP1 inhibitor (TPI)-based strategy shows that SWNTs can enter RAW macrophages with high uptake (>99%), and intracellularly deliver small molecule TPI inhibitors in a pH-dependent manner in vitro. At pH >7, the drug fairly stably interacts with the SWNTs via π-π electronic interactions, while under more acidic conditions (pH ˜6.5 and 5.5; endosome pH: ˜4.5 to 6.5; atherosclerotic plaques pH: ˜6.5 to 8.5), TPI release accelerates as π-π stacking interactions are pH sensitive given that cyclohexadienedione may protonate at acidic pH, which can increase aqueous solubility and interfere with π-π stacking interactions. TPI release increased more quickly in serum than in PBS, as expected due to the solubilizing effect of serum proteins on TPI (FIG. 19). We chose TPI because i) it is a particularly selective tyrosine phosphatase inhibitor toward SHP-1 versus similar proteins such as SHP-2, and ii) is an even more effective SHP-1 inhibitor than NSC87877 (with an IC₅₀ of 0.355 μM versus 40 nM for TPI) and sodium stibogluconate, a clinically-used SHP-1 inhibitor (by over two orders of magnitude).

Thus, we showed that macrophage phagocytosis can be stimulated intracellularly, by small molecules via the CD47-SIRPα signaling pathway, for the first time by pH-dependent drug release from the nanocarrier. Noting that the studies were conducted in different systems, the phagocytic index ratio of SWNT-TPI versus control (in this study) and anti-CD47 antibodies versus baseline control is 2.16 and 2.11, respectively. These data suggest the SWNT-TPI delivery approach stimulates macrophage efferocytosis in vitro at least as effectively as anti-CD47 antibodies. Unlike the anti-CD47 antibody approach, however, which necessarily involves a single injection at a time for a single treatment, our platform nanoparticle releases TPI over a period of days, and thus will likely require administration substantially less often than comparable protein-based approaches—in addition to its likely decreased toxicity and costs.

While SWNTs alone appear to slightly decrease proliferation, we reiterate that based on in-depth studies these very short, functionalized SWNTs are non-toxic in rodents and non-human primates, and indeed most control conditions decrease proliferation in such assays (e.g., IgG alone). We further note the biphasic release profile of TPI from SWNTs (FIG. 3), and we will reduce this pre-day 1 “mini-burst” release in order to make SWNT-TPI more reliable and reduce the administration dose. Our current study demonstrates that cell selective SWNTs can intracellularly deliver therapy to enhance macrophage efferocytosis with a low TPI dose selective to SHP1 in vitro.

We successfully prepared small molecule TPI-loaded SWNT nanodelivery vehicles using noncovalent π-π stacking interactions to intracellularly induce macrophage efferocytosis in vitro. The combination of selective uptake into inflammatory monocytes/macrophages and pH dependent drug release makes this therapy a novel, safe, and effective alternative to anti-CD47 antibodies and similar extracellular receptor blocking agents. This new mechanism can reduce atherosclerotic plaque formation and overall morbidity without the toxic side effects of antibodies due to its highly selective uptake, highly effective, SHP1-selective inhibitor, and long-term pH-dependent release.

### Methods

Preparation of SWNT-TPI. To prepare the nanocarrier (FIG. 16), HiPco (high-pressure carbon monooxide) RAW SWNTs were added to an aqueous solution of DSPE-050PA (NOF Corporation) and sonicated under ice bath conditions for ˜12 hrs, then centrifuged at 100,000g for 2 hr to obtain PEGylated SWNTs. Unbound surfactant was washed away with PBS using 100 kDa centrifugal filters (Millipore) at 3500 rpm/10 min. To conjugate Cy5.5 Mono NHS Ester (GE Life Sciences) to SWNT-PEG, Cy5.5 Mono NHS Ester was diluted in DMSO and small aliquots were incubated with SWNT-PEG solution (pH˜8.1) overnight. Excess Cy5.5 dye was removed by centrifugal filtration to obtain purified SWNT-Cy5.5 (SWNT), as described previously. SWNT concentrations were determined by Nanodrop (Thermo Scientific, Nanodrop2000) with an extinction coefficient of 7.9×10⁶ M-1 cm⁻¹ at 808 nm. To load TPI (2-(2,5-dichlorophenyl)-2,5-cyclohexadiene-1,4-dione) onto SWNTs, TPI dried powder was diluted in DMSO and added to stirred with SWNT-Cy5.5 (pH=7.4) at room temperature overnight to form SWNT-Cy5.5-TPI (SWNT-TPI). Unbound TPI was washed with PBS 5 times using 100 kDa centrifugal filters (Millipore) at 3500 rpm/10 in. The concentration of loaded TPI was measured using a Nanodrop at its absorption of 312 nm with a standard curve.

XPS sample preparation and analysis. The surface chemistry of the top 50-80 Å of SWNT preparations was evaluated via X-Ray Photoelectron Spectroscopy (XPS). The measurements were performed using a PHI 5400 ESCA system. The base pressure of the instrument was less than 10⁻⁸ Torr. All samples were freeze dried for 72 h, and a 1 cm2 sample was mounted onto the sample holder with double sided copper tape. The X-Ray was a non-monochromatic Mg source with a take-off angle of 45 degrees. Two types of scans were performed for each sample; a survey scan from 0-1100 eV taken with a pass energy of 187.85 eV and regional scans of each element at a pass energy of 29.35 eV. The data were fit using PHI Multipak (v8.0) software.

FTIR sample preparation and analysis. To prepare samples for Fourier Transform Infrared (FT-IR) spectroscopy assessment, all samples were freeze-dried for 72 h. They were then applied as a powder to take an FT-IR spectrum using ATR (attenuated total reflectance) mode on a PerkinElmer Spectrum One FT-IR spectrometer (PerkinElmer) operated in the range 4000-550 cm⁻¹ by accumulating 20 scans. UV-Vis spectroscopy sample preparation and analysis UV-Vis spectroscopy in the 200-800 nm region (nanodrop type) was performed for TPI, SWNT, SWNT-Cy5.5, and SWNT-TPI. We used 2 μL per sample for each test. Transmission electron microscopy 10 μL of 10 nM SWNT were drop casted onto ultrathin lacey carbon 400 mesh TEM grids (Ted Pella, Inc.). Transmission electron microscopy (TEM) (Titan TEM, Thermo Fisher Scientific) was then performed after sample dried using an acceleration voltage of 80 kV. For SWNT-PEG negative staining, 10 μL of 10 nM SWNTs were drop casted onto ultrathin lacey carbon 400 mesh TEM grids (Ted Pella, Inc.) and incubated for 10 minutes. The grids were then washed with ultrapure water and negatively stained with 1% uranyl acetate for 30 seconds and subsequently dried on Whatman grade 1 filter paper to conduct TEM characterization.

TPI release Assay. We conducted assays to evaluate TPI release from SWNTs in carbonate buffer (pH 9) and PBS buffer (pH 6.5 and 5.5). SWNT-TPI (200 μL, 16.5 TPI molecules/SWNT on average) was added to MINI Dialysis Devices (MWCO: 20K) and the volume of release medium was 3 mL. At fixed timepoints from 1 day to 7 days, 2 μL of the outlet medium was removed to test the rate of TPI release. The cumulative drug release (CDR) percentage was calculated by the following equation: CDR (%)=Wreleased/Wloaded×100% Here, CDR represents the cumulative drug release percentage, Wreleased represents the mass of released TPI in the release media, Wloaded represents the total mass of TPI. TPI release in serum (20% Fetal Bovine Serum (FBS) in Phosphate-buffered saline (PBS)): the timepoints, sample volume, and calculation methods were identical to the above.

Cell Culture RAW 264.7 cells (a murine macrophage cell line) were cultured in 10% Fetal Bovine Serum (FBS, Hyclone, USA) in Dulbecco's Modified Eagle Medium (DMEM, Gibco, USA) at 37°C., 5% CO₂. RAW cell uptake of SWNT assay 1-2×10⁶ RAW cells were placed in 10 cm tissue culture dish in 10% FBS in DMEM at 370 C, 5% CO2 for 24 hrs. SWNTs at 4 nM, and SWNT-TPI with TPI concentration of 32 and 80 nM (SWNTs at 4 nM) were respectively added to the RAW cells and cultured for 48 hrs. The cells were harvested and washed once with 1% bovine serum albumin (BSA, Gemini, USA)/Phosphate-Buffered Saline without calcium and magnesium (PBS, Corning) and analyzed by FACS RSL II (BD Bioscience, CA, USA). The data were analyzed by FlowJo10 (the Cy5.5 positive cells are recognized as the cells taking up SWNTs or SWNT-TPI). The rate of Cy5.5-positive cell uptake was calculated as Cy5.5 positive cells/total cells×100%.

Phagocytosis assay PBS, SWNTs at 4 nM, and SWNT-TPI with different TPI concentration (TPI: 32 and 80 nM, SWNTs at 4 nM) were respectively cultured with RAW cell phagocytes in 10% FBS in DMEM at 370 C, 5% CO2 for 48 hrs. The control RAW phagocytes were cultured in 10% FBS with DMEM for 48 hrs and then stained with 1 nM CellTracker Deep Red (Invitrogen, USA) for 30 mins. Target RAW cells were cultured with DMEM without FBS for 24 hrs, adding Staurosporine (Sigma, USA) at 1 nM for 3.5 hrs to induce apoptosis and staining with 1.25 nM CellTracker Orange CMRA (Invitrogen, USA) for 30 mins. 100,000 target RAW cells labeled with CellTracker Orange were loaded in each well of ultralow attachment 96 well plate (Costa, USA). 50,000 RAW phagocytes treated with PBS (labeled with deep red), SWNT, or SWNT-TPI (with Cy5.5) for 48 hrs were respectively added to target cells and mixed well in 0.1 mL of DMEM without FBS. The two cell types were co-cultured at 37 C0. 5% CO2 for 2 hrs. The mixed cells were washed once with 1% bovine serum albumin (BSA, Gemini, USA)/PBS and analyzed by FACS RSL II (BD Bioscience, CA, USA). The data were analyzed by FlowJo10. Double-positive cells were recognized as the target cells (orange color) engorged by phagocytes (deep red color or Cy5.5). The Phagocytosis index was calculated as double positive cells/(double positive cells+total phagocytes)×100%.

Cell Proliferation Assay. A modified MTT (3-[4,5-dimethyl-thiazol-2-yl]-2,5-diphenyltetrazolium bromide) assay was performed to analyze macrophage proliferation. RAW 264.7 cells were plated at 25,000 cells/well in a 96-well plate grown overnight at 37°C., and serum-starved for the next 24 hrs. Cells were stimulated with 10% FBS for 24 hrs with SWNTs (4 nM), SWNT-TPI (TPI: 25 nM and 50 nM), or TPI (25 nM), and then incubated with 10 μL of MTT AB solution (Millipore, Billerica) for 2 hr. 100 μL of acidic isopropanol (0.04N HCl) was added to each well and the absorbance was measured at 570 nm (reference wavelength 650 nm) on an ELISA plate reader a SpectraMax 190 Microplate Reader (Molecular Devices). Statistical analysis Statistical differences were determined by one-way ANOVA, and results were expressed as means±SE. p-value<0.05 and p-value<0.01 were considered to indicate statistical significance. Statistical analyses were performed using GraphPad Prism 7 (GraphPad Inc.).

What is claimed is:

1. A composition for delivering a therapeutic agent that increases efferocytosis in a mammalian subject, comprising:
single-walled carbon nanotubes (SWNTs) and a therapeutic agent.

2. The composition of claim 1, wherein the immune or inflammatory disorder is an atherosclerotic cardiovascular disease (ASCVD).

3. A composition for targeted delivery of a therapeutic agent that increases efferocytosis in a diseased tissue marked by the presence of Ly-6C^{hi} monocytes, comprising:
SWNTs;
the SWNTs deliver a therapeutic agent that increases efferocytosis for delivery to a diseased tissue marked by the presence of Ly-6C^{hi} monocytes.

4. The composition of claim 1 or claim 3, wherein the therapeutic agent increases efferocytosis of apoptotic cells or cellular degradation products by reversing an anti-phagocytic signal.

5. The composition of claim 1 or claim 3, wherein the therapeutic agent increases efferocytosis of cellular degradation products in atherosclerotic plaques.

6. The composition of claim 1 or claim 3, wherein the therapeutic agent is a small molecule.

7. The composition of claim 6, wherein the small molecule is an inhibitor of a molecule in a CD47/SIRPa signaling pathway.

8. The composition of claim 7, wherein the small molecule inhibits SHP1 phosphatase.

9. The composition of claim 8, wherein the small molecule is the SHP1 inhibitor TPI-1.

10. The composition of claim 8, wherein the small molecule that inhibits SHP1 is NSC-87877.

11. A method of treating an immune and/or inflammatory disorder in a subject, the method comprising:
administering to the subject an effective dose of the composition of claim 1 or claim 3, and treating the immune and/or inflammatory disorder.

12. The method of claim 11, wherein the immune and/or inflammatory disorder is ASCVD.

13. The method of claim 11 or claim 12, wherein the therapeutic agent increases efferocytosis of apoptotic cells or cellular degradation products by reversing an anti-phagocytic signal.

14. The method of claim 11 or claim 12, wherein the therapeutic agent increases efferocytosis of cellular degradation products in atherosclerotic plaques.

15. The method of claim 11 or claim 12, wherein the therapeutic agent is a small molecule.

16. The method of claim 15, wherein the small molecule is an inhibitor of a molecule in a CD47/SIRPa signaling pathway.

17. The method of claim 16, wherein the small molecule inhibits SHP1 phosphatase.

18. The method of claim 17, wherein the small molecule is the SHP1 inhibitor TPI-1.

19. The method of claim 17, wherein the small molecule that inhibits SHP1 is NSC-87877.

20. The method of claim 11 or claim 12, wherein the subject has been diagnosed as having at least one 9p21 risk allele for atherosclerosis.

21. The method of claim 11 or claim 12, further comprising the step of genotyping the subject for the presence of at least one 9p21 risk allele.

22. The method of claim 11 or claim 12, wherein the 9p21 risk allele is genotyped by determination of the presence of an SNP variant at 9p21 associated with risk.

23. The method of claim 11 or claim 12, wherein the subject is a mammal.

24. The method of claim 23, wherein the mammal is a human.

25. A kit for delivery of a therapeutic agent that increases efferocytosis to treat an immune and/or inflammatory disorder in a mammalian subject, comprising:
single-walled carbon nanotubes (SWNTs) comprising:
a therapeutic agent that increases efferocytosis for delivery to and treatment of an individual having an immune or inflammatory disorder; and
instructions for use.

26. Use of the composition of claim 1 or claim 3 in the manufacture of a medicament to stabilize, prevent or reduce atherosclerotic plaques, wherein the medicament is used to treat an immune and/or inflammatory disorder in a mammalian subject.
